(19) United States
(12) Patent Application Publication
(10) Pub. No.: US 2010/0291091 A1
(43) Pub. Date: Nov. 18, 2010

(54) CANCER ASSOCIATED GENE LY6K

(75) Inventors: Yusuke Nakamura, Tokyo (JP); Yataro Daigo, Tokyo (JP); Shuichi Nakatsuru, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ONCOTHERAPY SCIENCE ,inc., Kanagawa (JP)

(21) Appl. No.: 12/671,447

(22) PCT Filed: Nov. 21, 2007
(86) PCT No.: PCT/JP2007/001281

§ 371 Date: Jul. 6, 2010

Related U.S. Application Data
(60) Provisional application No. 60/952,830, filed on Jul. 30, 2007.

(51) Int. Cl. A61K 39/395; C12Q 1/68; G01N 33/566; G01N 33/68; A61K 31/7084; C07H 21/00; C07K 16/28; G01N 33/574; C40B 30/00; A61P 35/00

(52) U.S. Cl. 424/139.1; 435/6; 435/7.21; 435/7.23; 436/86; 514/44 A; 536/24.5; 530/389.6; 435/7.1; 436/501; 435/7.92; 435/7.94; 436/64; 506/7

(57) ABSTRACT

LY6K is identified herein as a potential biomarker useful for the diagnosis of cancer, such as lung and esophageal cancers, as well as for the prognosis of patients with these diseases. As discussed in detail herein, LY6K is specifically over-expressed in most lung and esophageal cancer tissues examined, and is elevated in the sera of a large proportion of patients with these tumors. Accordingly, LY6K may be used in combination with other tumor markers to significantly improve the sensitivity of cancer diagnosis. LY6K may be used in the treatment of ESCC cells, as demonstrated by the fact that small interfering RNAs (siRNAs) of LY6K suppressed growth of the cancer cells. Moreover, the LY6K molecule is also a likely candidate for development of novel therapeutic approaches, such as antibody therapy.

The present application claims the benefit of U.S. Provisional Application No. 60/952,830, filed Jul. 30, 2007, the entire disclosure of which is hereby incorporated herein by reference for all purposes.

### TECHNICAL FIELD

The present invention relates to methods for detecting, diagnosing, and providing a prognosis for cancer or a predisposition therefore, particularly esophageal and lung cancer. More particularly, the present invention relates to methods for detecting, diagnosing, and providing a prognosis of esophageal cancer (EC), for example, esophageal squamous-cell carcinoma (ESCC), and lung cancer, for example non-small cell lung cancer (NSCLC), as well as methods of treating and preventing esophageal and lung cancer.

### BACKGROUND ART

Aerodigestive tract cancer (including carcinomas of lung, esophagus, oral cavity, pharynx, and larynx) accounts for one-third of all cancer deaths in the United States and is the most common type of cancer in some areas of the world (Berwick M & Schantz S. Cancer Metastasis Rev. 1997 September-December;16(3-4):329-47.). Lung cancer is one of the most common malignant tumors in the world, and non-small cell lung cancer (NSCLC) accounts for nearly 80% of those cases (Jemal A, et al. CA Cancer J Clin. 2006 March-April;56(2):106-30.). Esophageal squamous-cell carcinoma (ESCC) is one of the most lethal malignancies of the digestive tract, and, at the time of diagnosis, most patients are already at an advanced stage (Shimada H, et al. Surgery. 2003 May;133(5):486-94.).

Despite modern surgical techniques combined with various adjuvant treatment modalities, such as radiotherapy and chemotherapy, the overall 5-year survival rate of ESCC patients still remains at 40-60% (Tamoto E, et al. Clin Cancer Res. 2004 Jun. 1;10(11):3629-38.), and that of lung cancer patients is only 15% (Parkin DM. Lancet Oncol. 2001 September;2(9):533-43., Naruke T, et al. Ann Thorac Surg. 2001 June;71(6):1759-64.). Moreover, most survivors experience a substantially reduced quality of life. Several tumor markers, such as progastrin-releasing peptide (ProGRP), neuron-specific enolase (NSE), cytokeratin 19-fragment (CYFRA 21-1), squamous-cell carcinoma antigen (SCC), and carcinoembryonic antigen (CEA), have been shown to be elevated in the serum of lung cancer patients (Rastel D, et al. Eur J Cancer. 1994;30A(5):601-6.), whereas SCC, CEA, and CYFRA 21-1 have been found to be elevated in the serum of advanced ESCC patients (Kawaguchi H, et al. Cancer. 2000 Oct. 1;89(7):1413-7.). However, their sensitivity remains at 20-50%, and no tumor marker has been found to be sufficiently useful in the detection of lung cancer and ESCC at potentially curative stage. Furthermore, a limited number of practical prognostic biomarkers is presently available for selection of treatment modalities for individual patients. Therefore, new diagnostic tools and therapeutic strategies, such as molecular-targeted agents, antibody therapy, and cancer vaccines, are urgently required (Naruke T, et al. Ann Thorac Surg. 2001 June;71(6):1759-64.).

Cancer-testis antigens (CTAs) are proteins that are highly expressed in cancer cells, but not in normal cells, with the exception of cells in reproductive tissues such as testis, ovary, and placenta (Boon T & Old L J. Curr Opin Immunol. 1997 Oct. 1;9(5):681-3., Scanlan M J, et al. Cancer Immun. 2004 Jan. 23;4:1.). Since the cells from these tissues do not express major histocompatibility complex (MHC) class I molecules, CTAs represent promising targets for immunotherapy and have potential as biomarker for diagnosis of cancer and monitoring of relapse.

Systematic analysis of expression levels of thousands of genes using cDNA microarray technology provides an effective approach for identifying molecules involved in pathways of carcinogenesis or those associated with the efficacy of anti-cancer therapy (Kakiuchi S, et al. Mol Cancer Res. 2003 May;1(7):485-99; Kikuchi T, et al. Oncogene. 2003 Apr. 10;22(14):2192-205; Kakiuchi S, et al. Hum Mol Genet. 2004 Dec. 15;13(24):3029-43. Epub 2004 Oct. 20; Kikuchi T, et al. Int J Oncol. 2006 April;28(4):799-805; Taniwaki M, et al. Int J Oncol. 2006 September;29(3):567-75; Yamabuki T, et al. Int J Oncol. 2006 June;28(6):1375-84.); some of such genes or their gene products have potential as target molecules for development of novel therapies and/or as cancer biomarkers.

To identify such molecules, particularly for CTAs, genome-wide expression profile analysis of 101 lung cancer and 19 ESCC patients, coupled with enrichment of tumor cells by laser-capture microdissection, was performed (Kikuchi T, et al. Oncogene. 2003 Apr. 10;22(14):2192-205; Kakiuchi S, et al. Hum Mol Genet. 2004 Dec. 15;13(24):3029-43. Epub 2004 Oct. 20; Kikuchi T, et al. Int J Oncol. 2006 April;28(4):799-805; Taniwaki M, et al. Int J Oncol. 2006 September;29(3):567-75; Yamabuki T, et al. Int J Oncol. 2006 June;28(6):1375-84.). The results were then compared with the expression profile data of 31 normal human tissues (27 adult and 4 fetal organs) (Saito-Hisaminato A, et al. DNA Res. 2002 Apr. 30;9(2):35-45; Ochi K, et al. J Hum Genet. 2003;48(4):177-82. Epub 2003 Feb. 21.).

To verify the biomedical and clinicopathological significance of the respective gene products, a screening system was established using a combination of the tumor-tissue microarray analysis of clinical lung and esophageal cancer materials and RNA interference (RNAi) techniques (Suzuki C, et al. Cancer Res. 2003 Nov. 1;63(21):7038-41; Ishikawa N, et al. Clin Cancer Res. 2004 Dec. 15;10(24):8363-70; Kato T, et al. Cancer Res. 2005 Jul. 1;65(13):5638-46; Furukawa C, et al. Cancer Res. 2005 Aug. 15;65(16):7102-10; Ishikawa N, et al. Cancer Res. 2005 Oct. 15;65(20):9176-84; Suzuki C, et al. Cancer Res. 2005 Dec. 15;65(24):11314-25; Ishikawa N, et al. Cancer Sci. 2006 August;97(8):737-45; Takahashi K, et al. Cancer Res. 2006 Oct. 1;66(19):9408-19; Hayama S, et al. Cancer Res. 2006 Nov. 1;66(21):10339-48; Kato T, et al. Clin Cancer Res. 2007 Jan. 15;13(2 Pt 1):434-42; Suzuki C, et al. Mol Cancer Ther. 2007 February;6(2):542-51; Yamabuki T, et al. Cancer Res. 2007 Mar. 15;67(6):2517-25.).

Recent acceleration in identification and characterization of novel molecular targets for cancer therapy has enhanced development of new types of anticancer agents, antibodies and vaccines (Kawaguchi H, et al. Cancer. 2000 Oct. 1;89(7):1413-7.). Molecular-targeted drugs are expected to be highly specific to malignant cells and, due to their well-defined mechanisms of action, have minimal adverse effects. As an approach to such a goal, one promising strategy combines the power of genome-wide expression analysis to effectively screen genes that are over-expressed in cancer cells but scarcely expressed in normal organ tissues, with high throughput screening of their protein expression related to clinical outcome by means of tissue microarray as well as with examining loss of function phenotypes by RNAi systems (Suzuki C, et al. Cancer Res. 2003 Nov. 1;63(21):7038-41; Ishikawa N, et al. Clin Cancer Res. 2004 Dec. 15;10(24):8363-70; Kato T, et al. Cancer Res. 2005 Jul. 1;65(13):5638-46; Furukawa C, et al. Cancer Res. 2005 Aug. 15;65(16):7102-10; Ishikawa N, et al. Cancer Res. 2005 Oct. 15;65(20):9176-84; Suzuki C, et al. Cancer Res. 2005 Dec. 15;65(24):11314-25; Ishikawa N, et al. Cancer Sci. 2006 August;97(8):737-45; Takahashi K, et al. Cancer Res. 2006 Oct. 1;66(19):9408-19; Hayama S, et al. Cancer Res. 2006 Nov. 1;66(21):10339-48; Kato T, et al. Clin Cancer Res. 2007 Jan. 15;13(2 Pt 1):434-42; Suzuki C, et al. Mol Cancer Ther. 2007 February;6(2):542-51; Yamabuki T, et al. Cancer Res. 2007 Mar. 15;67(6):2517-25.). Using this combination approach, LY6K was herein discovered to be a novel cancer testis antigen (CTA) whose over-expression not only affect the growth of the cancer cells but also correlates with an unfavorable prognostic significance in NSCLC patients

LY6K was initially identified by several groups (Accession No. AJ001348; AB 105187; SEQ ID NO: 2 encoded by SEQ ID NO: 1) as an unannotated transcript. More recent analysis by bioinformatics classified it as a member belonging to the LY6 family having a high homology to the low molecular-weight GPI-anchored molecule (de Nooij-van Dalen A G, et al. Int J Cancer. 2003 Mar. 1;103(6):768-74.). Like others in the LY6 family, LY6K has 10 cysteine residues in a conserved position and harbors the sequence structure that, in theory, determines GPI anchoring. Members of the LY6 family believed to possess functions related to cell signaling and/or cell adhesion (Bamezai A & Rock K L. Proc Natl Acad Sci U S A. 1995 May 9;92(10):4294-8.), although the precise role of LY6K in lung carcinogenesis or its physiological function in normal cells is presently unknown. Since the LY6K gene is located at chromosome 8q24, a region of allelic gain in more than half of lung cancers (Balsara B R, et al. Cancer Res. 1997 Jun. 1;57(11):2116-20.), its over-expression may result from amplification or chromosomal aberration at this locus.

GPI-anchored proteins are extracellular proteins anchored in the lipid bilayer surface of plasma membrane by GPI (McConville M J & Menon A K. McConville M J & Menon A K. Mol Membr Biol. 2000 January-March;17(1):1-16.). There are several known GPI-anchored proteins that are applicable to diagnosis of human cancer in certain clinical or pre-clinical settings. Human carcinoembryonic antigen (CEA) is presumed to be such a GPI-anchored protein (GOLD P & FREEDMAN S O. J Exp Med. 1965 Mar. 1;121:439-62.), and is highly expressed in a significant proportion of relatively advanced adenocarcinomas, particularly those from the colon, pancreas, breast, and lung (Hammarstrom S. Semin Cancer Biol. 1999 April;9(2):67-81.). Its presence in the serum of cancer patients has been used for disease staging and as an indicator of residual disease and/or tumor recurrences (Hammarstrom S. Semin Cancer Biol. 1999 April;9(2):67-81.). In addition, some tumor-specific markers and prognostic markers, such as CD109, glypican-3 (GPC3), CEA-related cell adhesion molecule 6 (CEACAM6), and prostate stem cell antigen (PSCA) are also categorized as GPI-anchored proteins (Hashimoto M, et al. Oncogene. 2004 Apr. 29;23(20):3716-20; Nakatsura T, et al. Biochem Biophys Res Commun. 2003 Jun. 20;306(1):16-25; Jantscheff P, et al. J Clin Oncol. 2003 Oct. 1;21(19):3638-46; Reiter R E, et al. Proc Natl Acad Sci USA. 1998 Feb. 17;95(4):1735-40.). Among them, CD109 and GPC3 are also known to be the cancer-testis antigens. In addition, there are several reports of GPI-anchored proteins acting as an immunotherapeutic target for human cancer. The CEA-TRICOM vaccines, designated as “TRICOM” for its inclusion of the three T-cell co-stimulatory molecules B7-1, ICAM-1, and LFA-3, has been shown to safely generate significant CEA-specific immune responses against advanced cancer in phase I clinical trials (Marshall J L, et al. J Clin Oncol 2005;23:720-31.). Recently, two independent studies demonstrated that a passive immunotherapy approach using an anti-PSCA monoclonal antibody inhibited prostate tumor growth and metastasis formation, and further prolonged survival times of mice bearing human prostate cancer xenografts (Ross S, et al. Cancer Res 2002;62:2546-53; Saffran D C, et al. Proc Natl Acad Sci U S A 2001;98:2658-63.).

Thus, while elevated expression of LY6K mRNA in human head-and-neck squamous-cell carcinomas and breast cancers has been previously described (de Nooij-van Dalen A G, et al. Int J Cancer. 2003 Mar. 1;103(6):768-74., Lee J W, et al. Oncol Rep. 2006 December;16(6):1211-4.), no report to date has clarified the significance of the activation of LY6K in human cancer progression and its potential as a therapeutic target and serological/prognostic biomarker.

### SUMMARY OF THE INVENTION

In view of the above, it is an objective of the present invention is to provide a method for detecting cancer, diagnosing cancer, monitoring a course of treatment or providing a prognosis for cancer, or determining a predisposition to cancer, more particularly lung cancer (LC, e.g., NSCLC) and/or esophageal cancer (EC, e.g., ESCC), in a subject. To that end, by systematically analyzing the expression levels of thousands of genes with cDNA microarray technology, it was herein revealed that lymphocyte antigen 6 complex, locus K (referred to as “LY6K”; also known as “HSJ001348”, a cDNA for differentially expressed CO16 gene), a member of the LY6 family, appears to be a novel CTA that is commonly over-expressed in primary NSCLCs and ESCCs and is essential for the growth and/or survival of cancer cells.

Thus, the present invention provides a method for detecting, diagnosing, monitoring the course of treatment, providing a prognosis, or determining a predisposition for cancer, particularly lung cancer (LC, e.g., NSCLC) and/or esophageal cancer (EC, e.g., ESCC) in a subject by determining the expression level of the LY6K gene in a biological sample from a patient, for example, a solid tissue or bodily fluid sample. An increase in the expression level of LY6K detected in a test sample as compared to a normal control level indicates that the subject (from which the test sample was obtained) suffers from or is at risk of developing LC and/or EC.

The present invention further provides a cancer assay that combines both LY6K and CEA/CYFRA 21-1 to increase the sensitivity for the patients with LC or EC without changing the level of false diagnosis found in healthy volunteers.

The present invention also provides kits for detecting cancer, such as lung cancer or esophageal cancer, such kits including (i) an immunoassay reagent for determining the level of LY6K in a patient derived sample, such as a blood sample; and (ii) a positive control sample for LY6K. The kits may further include either or both of (iii) an immunoassay reagent for determining the level of CEA in a patient sample and a positive control sample for CEA, and (iv) an immunoassay reagent for determining the level of CYFRA 21-1 in a patient sample and a positive control sample for CYFRA 21-1.

The present invention further provides methods of identifying agents that inhibit the expression or activity of LY6K by contacting a test cell expressing LY6K with a test compound and determining the level of LY6K expression or the activity of its gene product. The test cell can be an epithelial cell, for example, an epithelial cell obtained from an esophageal squamous-cell carcinoma. A decrease in the level of expression of LY6K or in the level of the activity of its gene product as compared to an expression or activity level measured in the absence of the agent indicates that the test agent is an inhibitor of LY6K and can be used to reduce a symptom of cancer, particularly LC or EC.

The present invention also provides a kit that include a detection reagent which binds to LY6K nucleic acids or polypeptides. Also provided is an array of nucleic acids that binds to LY6K.

Therapeutic methods of the present invention include methods of treating or preventing cancer, particularly LC and/or EC, in a subject including the step of administering to the subject a composition containing one or more antisense oligonucleotides. In the context of the present invention, the antisense composition should be capable of reducing the expression of LY6K. Accordingly, the antisense composition can contain one or more nucleotides which are complementary to LY6K sequences, SEQ ID NO: 1.

Alternatively, the present methods can include the step of administering to a subject a composition containing one or more small interfering RNA (siRNA) oligonucleotides. In the context of the present invention, the siRNA composition should be capable of reducing the expression of LY6K nucleic acids. Examples of siRNA against a Homo sapiens lymphocyte antigen 6 complex, locus K (LY6K) (SEQ ID NO; 1, 2) suitable for inhibiting proliferation and viability of lung cancer cells and/or esophageal cancer cells are described herein. Thus, in some embodiments of the present invention, LY6K serves a therapeutic target for lung cancer and/or esophageal cancer.

The present invention also provides vaccines and vaccination methods. For example, methods of treating or preventing cancer, for example LC and/or EC, in a subject may involve administering to the subject a vaccine composition composed of LY6K polypeptides or immunologically active fragments of such polypeptides. In the context of the present invention, an immunologically active fragment is a polypeptide that is shorter in length than the full-length, naturally-occurring protein yet which is sufficient to induce an immune response analogous to that induced by the full-length protein. For example, an immunologically active fragment is in most cases at least 8 residues in length and capable of stimulating an immune cell including, a T cell or a B cell. Immune cell stimulation can be measured by detecting cell proliferation, elaboration of cytokines (e.g., IL-2), or production of an antibody. See, for example, Harlow and Lane, Using Antibodies: A Laboratory Manual, 1998, Cold Spring Harbor Laboratory Press; and Coligan, et al., Current Protocols in Immunology, 1991-2006, John Wiley & Sons.

One advantage of the methods described herein is that cancer, particularly lung cancer and/or esophageal cancer, can be identified at a very early and potentially curative stage, generally prior to detection of overt clinical symptoms.

Regarding the specific aims and objectives recited above, it will be understood by those skilled in the art that one or more aspects of this invention can meet certain objectives, while one or more other aspects can meet certain other objectives. Each objective may not apply equally, in all its respects, to every aspect of this invention. As such, the objects herein can be viewed in the alternative with respect to any one aspect of this invention. Additional objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments which follows:

FIG. 1. depicts the expression and subcellular localization of LY6K in NSCLCs and ESCCs. Part A depicts the expression of LY6K in 10 clinical NSCLC samples examined by semiquantitative RT-PCR analysis. Part B depicts the expression of LY6K in 8 clinical ESCC samples, as detected by semi-quantitative RT-PCR analysis. Part C depicts the expression of LY6K protein in four representative pairs of NSCLC samples (left panels) and four lung-cancer cell lines (right panels), examined by western-blot analysis. Part D, (left panels) depicts the subcellular localization of endogenous LY6K protein in lung-cancer cells. LY6K is stained at the cytoplasm of the cell with granular appearance in LC319 and NCI-H1373 cells, but not in NCI-H226 and A427 cells(right panels). Measurement of secreted LY6K levels with ELISA in culture medium of LY6K-expressing LC319 and NCI-H1373 cells, and non-expressing NCI-H226 and A427 cells.

FIG. 2. depicts the expression of LY6K in normal organ tissues as well as lung SCC tissues. In Part A, results of Northern blot analysis of the LY6K transcript in 23 normal adult human tissues are depicted. In Part B, results of immunohistochemical evaluation of LY6K protein in representative normal tissues; adult heart, liver, lung, kidney, and testis, as well as lung SCC tissues, are depicted.

FIG. 3. depicts the association of LY6K over-expression with poor clinical outcomes for NSCLC and ESCC patients. In Part A, results of immunohistochemical evaluation of LY6K expression on tumor tissue microarrays are depicted (upper panels, X100; lower panels, X200). Examples are shown of strong, weak, and absent LY6K expressions in cancer tissues, and of no expression in normal tissues; lung SCC and normal lung. In Part B, results of Kaplan-Meier analysis of survival of patients with NSCLC are depicted (P=0.0026 by the Log-rank test). In Part C, results of immunohistochemical evaluation of LY6K expression on tumor tissue microarrays are depicted (upper panels, X100; lower panels, X200). Examples are shown of strong, weak, and absent LY6K expressions in cancer tissues, and of no expression in normal tissues; ESCC and normal esophagus. In Part D, results of Kaplan-Meier analysis of survival of patients with ESCC (P=0.0455 by the Log-rank test) according to the expression levels of LY6K are depicted.

FIG. 4. depicts the serologic concentration of LY6K determined by ELISA in serum of patients with lung cancers or esophageal cancers and in healthy controls or non-neoplastic lung-disease patients with COPD. In Part A, the distribution of LY6K in sera from patients with lung ADC, lung SCC, and ESCC is depicted. Averaged serum levels are shown under the panel. Differences were significant between lung ADC patients and healthy individuals (P<0.0001, Mann-Whitney U test), between lung SCC patients and healthy individuals (P=0.0145) and between ESCC patients and healthy individuals (P<0.0001). In Part B, the distribution of LY6K in sera from patients at various clinical stages of lung cancers and esophageal cancers is depicted.

FIG. 5. depicts the serologic concentration of LY6K, CEA and CYFRA 21-1 determined by ELISA in serum of patients with lung cancers or esophageal cancers. In Part A, (left panel) ROC curve analysis of LY6K and CEA as serum markers for NSCLC is depicted (X-axis, 1-specificity; Y-axis, sensitivity). The cut-off level was set to provide optimal diagnostic accuracy and likelihood ratios (minimal false negative and false positive results) for LY6K, i.e., 157.0 pg/ml. (right panel) Relationship between serum levels of LY6K and CEA (X-axis, LY6K concentration; Y-axis, CEA concentration). In Part B, (left panel) ROC curve analysis of LY6K and CYFRA 21-1 as serum markers for NSCLC is depicted (X-axis, 1-specificity; Y-axis, sensitivity). The cut-off level was set to provide optimal diagnostic accuracy and likelihood ratios (minimal false negative and false positive results) for LY6K, i.e., 157.0 pg/ml. (right panel) Relationship between serum levels of LY6K and CYFRA 21-1 (X-axis, LY6K concentration; Y-axis, CYFRA 21-1 concentration).

FIG. 6. depicts the relationship among three serum markers in NSCLCs. Part A (left panel) depicts the relationship between serum levels of CEA and CYFRA 21-1 for NSCLC patients (X-axis, CEA concentration; Y-axis, CYFRA 21-1 concentration), (middle panel) the relationship between serum levels of LY6K and CEA (X-axis, LY6K concentration; Y-axis, CEA concentration), and (right panel) the relationship between serum levels of LY6K and CYFRA 21-1 (X-axis, LY6K concentration; Y-axis, CYFRA 21-1 concentration). In Part B, combinations of CEA, CYFRA 21-1 and LY6K for NSCLC diagnosis are depicted.

FIG. 7. depicts the relationship among three serum markers in ESCCs. Part A (left panel) depicts the relationship between serum levels of CEA and CYFRA 21-1 for ESCC patients (X-axis, CEA concentration; Y-axis, CYFRA 21-1 concentration), (middle panel) the relationship between serum levels of LY6K and CEA (X-axis, LY6K concentration; Y-axis, CEA concentration), and (right panel) the relationship between serum levels of LY6K and CYFRA 21-1 (X-axis, LY6K concentration; Y-axis, CYFRA 21-1 concentration). In Part B, combinations of CEA, CYFRA 21-1 and LY6K for ESCC diagnosis are depicted.

FIG. 8. depicts the serologic concentration of LY6K determined by ELISA in serum of patients with lung cancers or esophageal cancers. Part A depicts serologic concentration of LY6K before and after surgery (postoperative days at 2 months) in patients with NSCLC and ESCC. A dotted line indicates the cut-off level for LY6K (157.0 pg/ml). Part B depicts serum LY6K levels (pg/ml) and the expression levels of LY6K in primary tumor tissues in the same NSCLC patients. ‘Score’ for tumor tissue indicates the intensity of LY6K staining that was evaluated using the criteria described in Materials and Methods.

FIG. 9. depicts the growth inhibition of NSCLC cells by siRNA against LY6K. Response of RERF-LC-AI cells or TE8 cells to si-LY6K-1 and -2, or control siRNAs (EGFP or SCR). In Part A, the level of LY6K protein expression detected by western-blot analysis in RERF-LC-AI cells treated with either control or si-LY6Ks is depicted. In Part B, colony-formation assays using RERF-LC-AI cells transfected with si-LY6K-1 and -2,-EGFP, or -SCR are shown. In Part C, the effect of siRNA against LY6K on cell viability, detected by MTT assays, is depicted. All assays were performed three times, and in triplicate wells. In Part D, the level of LY6K protein expression detected by western-blot analysis in TE8 cells treated with either control or si-LY6Ks is depicted. In Part E, the effect of siRNA against LY6K on cell viability, detected by MTT assays, is depicted. All assays were performed three times, and in triplicate wells.

### DETAILED DESCRIPTION OF THE INVENTION

It is to be understood that the present invention is not limited to the specific methodologies and protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims. It must be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

In the context of the present invention, the phrase “control level” refers to an mRNA or protein expression level detected in a control sample and may include any of (a) a normal control level, (b) a lung cancer specific control level and (c) an esophageal cancer specific control level. A control level can be a single expression pattern from a single reference population or composed from a plurality of expression patterns. For example, in the context of the present invention, the control level can be a database of expression patterns from previously tested cells. The phrase “normal control level” refers to a level of gene expression detected in a normal, healthy individual or in a population of individuals known not to be suffering from cancer, such as lung cancer or esophageal cancer. A normal individual is one with no clinical symptoms of cancer, either lung cancer or esophageal cancer. On the other hand, an “LC control level” or “EC control level” refers to a level of gene expression found in a population suffering from lung cancer or esophageal cancer, respectively.

Alternatively, a similarity in LY6K expression levels between a test sample and an the LC or EC control indicates that the subject (from which the test sample was obtained) suffers from or is at risk of developing LC or EC, respectively.

According to the present invention, an expression level of a particular gene is deemed “increased” when expression of the gene is increased by at least 0.1, at least 0.2, at least 1, at least 2, at least 5, or at least 10 or more fold as compared to a control level. LY6K gene expression can be determined by detecting mRNA of LY6K from a tissue sample from a patient, e.g., by RT-PCR or Northern blot analysis, or detecting a protein encoded by LY6K, e.g., by immunohistochemical analysis of a tissue sample from a patient.

In the context of the present invention, the tissue sample from a patient may be any tissue obtained from a test subject, e.g., a patient known to or suspected of having cancer, more particularly LC or EC. For example, the tissue can contain epithelial cells. More particularly, the tissue can be epithelial cells from non-small cell lung carcinoma or esophageal squamous-cell carcinoma.

Additional definitions are interspersed in the subsequent text, where applicable.

Overview

To identify novel biomarkers and therapeutic targets for cancer, particularly lung and esophageal cancers, genes that were highly transactivated in a large proportion of non-small cell lung carcinomas (NSCLCs) and esophageal squamous-cell carcinomas (ESCCs) were screened using a cDNA microarray representing 27,648 genes. A member of low molecular weight, GPI-anchored molecule-like protein, lymphocyte antigen 6 complex, locus K (LY6K) was selected as a candidate. Tumor-tissue microarray was applied to examine expression of LY6K protein in archival cancer samples from 413 NSCLC and 271 ESCC patients. Serum LY6K levels of 112 lung-cancer patients, 81 ESCC patients, and 74 healthy controls were measured by ELISA. The role of LY6K in cancer cell growth and/or survival was then examined by small interfering RNA (siRNA) experiments.

LY6K is abundantly expressed in the great majority of lung and esophageal cancers, while its expression is detected only in testis among normal tissues. A high level of LY6K expression is also associated with poor prognosis of patients with NSCLC (P=0.0026) as well as ESCC (P=0.0455), and multivariate analysis confirms its independent prognostic value for NSCLC (P=0.0201). In fact, the proportion of the serum LY6K-positive cases was 33.9% of NSCLC and 32.1% of ESCC, while only 4.1% of healthy volunteers were falsely diagnosed as positive. Furthermore, a combined assay, using both LY6K and carcinoembryonic antigen (CEA), judged 64.7% of the lung adenocarcinoma patients as positive while 9.5% of healthy volunteers were falsely diagnosed.

CEA is a glycoprotein involved in cell adhesion. It is normally produced during fetal development; however, the production of CEA stops before birth. Therefore, it is not usually found in the blood of healthy adults, although levels are raised in heavy smokers. Furthermore, serum from individuals with colorectal, gastric, pancreatic, lung and breast carcinomas have been shown to possess higher levels of CEA than healthy individuals. However, CEA results cannot be interpreted as absolute evidence confirming the presence or absence of malignant disease, but must be used in conjunction with information from other test procedures and from clinical evaluations of the patient tested. While CEA levels are elevated in smokers; patients with inflammation including infections, inflammatory bowel disease, and pancreatitis; some patients with hypothyroidism; cirrhosis; and in some patients with noncolorectal neoplasms especially gastric, pancreatic, breast, and ovarian, it cannot be considered a suitable screening test for occult cancer. Many negatives occur in patients with early carcinoma, and even in some patients with metastatic colorectal and other neoplasms. Therefore, markers that would improve the sensitivity of the assay, particularly in the context of diagnosing esophageal cancer are in great demand. As disclosed herein, LY6K is an example of such a sensitivity improving marker.

As demonstrated herein, the use of both LY6K and cytokeratin 19-fragment (CYFRA 21-1) increased assay sensitivity in the detection lung squamous-cell carcinomas up to 70.4%, while false positive rate were only 6.8%. CYFRA 21-1 measures soluble cytokeratin-19 fragments in serum and is a useful marker for lung carcinoma, especially squamous cell carcinoma. In addition, treatment of NSCLC cells with siRNAs against LY6K knocked-down its expression and resulted in growth suppression of the cancer cells. This data suggests that a cancer-testis antigen LY6K should be useful as a diagnostic/prognostic biomarker and probably as a therapeutic target for lung and esophageal cancers.

In sum, the present invention demonstrates that lymphocyte antigen 6 complex, locus K (LY6K) (Accession No. AJ001348; AB105187; SEQ ID NO: 2 encoded by SEQ ID NO: 1) is a cancer-testis antigen having potential as a biomarker for diagnosis of cancers such as lung and esophageal cancers as well as for assessing and monitoring patients with these diseases. Since serum levels of LY6K are shown herein to elevated in the sera of a large proportion of the patients, LY6K, combined with other tumor markers, can significantly improve the sensitivity of cancer diagnosis. It may also find utility as an initial diagnostic for identifying patients who might benefit from early systemic treatment. Moreover, LY6K, as an essential contributor to a growth-promoting pathway and to aggressive features of NSCLC and ESCC, is a likely target for development of therapeutic approaches, such as molecular-targeted drugs and immunotherapies for any types of cancers over-expressing this molecule.

Diagnosing Lung Cancer and Esophageal Cancer

The expression of the LY6K gene was found to be specifically elevated in patients with lung cancer or esophageal cancer. Therefore, the gene identified herein, as well as its transcription and translation products, find diagnostic utility as a marker for cancer. More particularly, by measuring the expression of the LY6K gene in a cell sample, lung cancer or esophageal cancer can be diagnosed. Thus, the present invention provides a method for diagnosing lung cancer or esophageal cancer or a predisposition for developing lung cancer or esophageal cancer in a subject by determining the expression level of the LY6K gene in the subject.

According to the present invention, an intermediate result for examining the condition of a subject may be provided. Such intermediate result may be combined with additional information to assist a doctor, nurse, or other practitioner to determine that a subject suffers from lung cancer or esophageal cancer. Alternatively, the present invention may be used to detect cancerous cells in a subject-derived tissue, and provide a doctor with useful information to determine that the subject suffers from lung cancer or esophageal cancer.

The diagnostic method of the present invention involves the step of determining (e.g., measuring) the expression of an LY6K gene. Using sequence information provided by the GenBank™ database entries for known sequences, the LY6K gene can be detected and measured using conventional techniques well known to one of ordinary skill in the art. For example, sequences within the sequence database entries corresponding to the LY6K gene can be used to construct probes for detecting RNA sequences corresponding to the LY6K gene in, e.g., Northern blot hybridization analyses. Hybridization probes typically include at least 10, at least 20, at least 50, at least 100, or at least 200 consecutive nucleotides of an LY6K sequence. As another example, the sequences can be used to construct primers for specifically amplifying the LY6K nucleic acid in, e.g., amplification-based detection methods, for example, reverse-transcription based polymerase chain reaction. As another example, an antibody against LY6K, e.g., an anti-LY6K polyclonal antibody or anti-LY6K monoclonal antibody, can be used for immunoassay, for example, immunohistochemical analysis, western blot analysis or ELISA, etc.

The level of the LY6K gene expression detected in a test cell population, e.g., a tissue sample from a patient, can then be compared to the expression level(s) of the gene in a reference cell population. The reference cell population may include one or more cells for which the compared parameter is known, i.e., non-small lung cancer cells (e.g, LC cells), esophageal squamous-cell carcinoma cells (e.g., EC cells), normal lung epithelial cells (e.g., non-LC cells) or normal esophageal epithelial cells (e.g., non-EC cells).

Whether or not a level of gene expression in a test cell population as compared to a reference cell population indicates the presence of LC, EC or a predisposition thereto depends upon the composition of the reference cell population. For example, if the reference cell population is composed of non-LC cells or non-EC cells, a similarity in gene expression level between the test cell population and the reference cell population indicates the test cell population is non-LC or non-EC. Conversely, if the reference cell population is made up of LC cells or EC cells, a similarity in gene expression between the test cell population and the reference cell population indicates that the test cell population includes LC cells or EC cells.

A level of expression of an LY6K gene in a test cell population is considered “altered” or deemed to “differ” if it varies from the expression level of the LY6K gene in a reference cell population by more than 1.1, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid can thus be used to normalize signal levels in the test and reference cell populations. Exemplary control genes include, but are not limited to, e.g., beta actin, glyceraldehyde 3-phosphate dehydrogenase and ribosomal protein P1.

The test cell population can be compared to multiple reference cell populations. Each of the multiple reference cell populations can differ in the known parameter. Thus, a test cell population can be compared to a first reference cell population known to contain, e.g., LC cells or EC cells, as well as a second reference cell population known to contain, e.g., non-LC cells or non-EC cells (normal cells). The test cell population can be included in a tissue or cell sample from a subject known to contain, or suspected of containing, LC cells or EC cells.

The test cell population can be obtained from a bodily tissue or a bodily fluid, e.g., biological fluid (for example, blood, sputum, saliva). For example, the test cell population can be purified from lung tissue or esophageal tissue. Preferably, the test cell population comprises an epithelial cell. The epithelial cell is preferably from a tissue known to be or suspected to be a non-small cell carcinoma or an esophageal squamous-cell carcinoma.

Cells in the reference cell population are preferably from a tissue type similar to that of the test cell population. Optionally, the reference cell population is a cell line, e.g. an LC cell line or an EC cell line (i.e., a positive control) or a normal non-LC cell line or a non-EC cell line (i.e., a negative control). Alternatively, the control cell population can be from a database of molecular information obtained from cells for which the assayed parameter or condition is known.

The subject is preferably a mammal. Exemplary mammals include, but are not limited to, e.g., a human, non-human primate, mouse, rat, dog, cat, horse, or cow.

Expression of the LY6K gene disclosed herein can be determined at the protein or nucleic acid level, using methods known in the art. For example, Northern hybridization analysis, using probes which specifically recognize one or more of these nucleic acid sequences, can be used to determine gene expression. Alternatively, gene expression can be measured using reverse-transcription-based PCR assays, using primers specific for the LY6K gene sequence e.g., SEQ ID NO: 1 and 2. Expression can also be determined at the protein level, i.e., by measuring the level of a polypeptide encoded by an LY6K gene, or the biological activity thereof. Such methods are well known in the art and include, but are not limited to, e.g., immunoassays that utilize antibodies to proteins encoded by the genes, e.g., anti-LY6K polyclonal antibodies which recognized amino acid sequence comprising SEQ ID NO: 18 or 19 described in Example 1, but not limited. The biological activities of the proteins encoded by the genes are generally well known and include, e.g., cell proliferative activity. See, Sambrook and Russell, Molecular Cloning: A Laboratory Manual, 3^{rd} Edition, 2001, Cold Spring Harbor Laboratory Press; Ausubel, Current Protocols in Molecular Biology, 1987-2006, John Wiley and Sons; and Harlow and Lane, Using Antibodies: A Laboratory Manual, 1998, Cold Spring Harbor Laboratory Press.

In the context of the present invention, EC or LC may be diagnosed by measuring the expression level of LY6K nucleic acids in a test population of cells, (i.e., a biological sample from a patient). Preferably, the test cell population contains an epithelial cell, e.g., a cell obtained from lung tiassue or esophageal tissue. Gene expression can also be measured from blood or other bodily fluids, for example, saliva or sputum. Other biological samples can be used for measuring protein levels. For example, the protein level in blood or serum from a subject to be diagnosed can be measured by immunoassay or other conventional biological assay.

Expression of the LY6K gene is first determined in the test cell population or biological sample and then compared to the normal control expression level of the LY6K gene. A normal control level corresponds to an expression of the LY6K gene typically found in a cell population from a subject known not to be suffering from LC or EC. An alteration or difference (e.g., an increase) in the level of expression of the LY6K gene in a tissue sample from a patient in comparison to expression from a normal control sample indicates that the subject is suffering from or is at risk of developing LC or EC. For example, an increase in the expression of the LY6K gene in the test cell population as compared to the expression in a normal control cell population indicates that the subject is suffering from or is at risk of developing LC or EC.

An increase in expression levels of the LY6K gene in the test cell population as compared to normal control expression levels indicates that the subject suffers from or is at risk of developing LC or EC. For example, increase in expression levels of at least 1%, at least 5%, at least 25%, at least 50%, at least 60%, at least 70%, at least 80%, at least 90% or more of the level of the LY6K gene indicates that the subject suffers from or is at risk of developing LC or EC.

Screening Assays

Identifying Agents that Inhibit LY6K Gene Expression:

An agent that inhibits the expression of the LY6K gene or the activity of its gene product can be identified by contacting a test cell population that expresses the LY6K gene with a test agent and then determining the subsequent level of gene expression or activity of its gene product. A decrease in the level of gene expression or of activity of its gene product in the presence of the agent as compared to the expression or activity level in the absence of the test agent indicates that the agent is an inhibitor of the LY6K gene and therefore useful in inhibiting LC and EC.

The test cell population can include any cells expressing the LY6K gene. For example, the test cell population can contain epithelial cells, for example, cells from lung tissue or esophageal tissue. Furthermore, the test cell population can be an immortalized cell line from a non-small lung cancer cell or an esophageal squamous-cell carcinoma cell. Alternatively, the test cell population can be composed of cells which have been transfected with the LY6K gene or which have been transfected with a regulatory sequence (e.g., promoter sequence) from the LY6K gene operably linked to a reporter gene.

The agent can be, for example, an inhibitory oligonucleotide (e.g., an antisense oligonucleotide, an siRNA or a ribozyme), an antibody, a polypeptide or a small organic molecule. Screening for suitable inhibitory agents can be carried out using high throughput methods, by simultaneously screening a plurality of agents using multiwell plates (e.g., 96-well, 192-well, 384-well, 768-well, 1536-well). Automated systems for high throughput screening are commercially available from, for example, Caliper Life Sciences, Hopkinton, Mass. Small organic molecule libraries available for screening can be purchased, for example, from Reaction Biology Corp., Malvern, Pa.; TimTec, Newark, Del.

Identifying Therapeutic Agents:

The differentially expressed LY6K gene disclosed herein can also be used to identify candidate therapeutic agents for treating LC and EC. The methods of the present invention therefore involve the screening a candidate therapeutic agent to determine if the test agent can convert an expression level of the LY6K gene that is characteristic of an LC state or an EC state to a gene expression level characteristic of a non-LC state or a non-EC state.

In the context of the instant method, a test cell population is exposed to a test agent or a plurality of test agents (sequentially or in combination) and the expression of the LY6K gene in the cells is measured. The expression level of the gene assayed in the test cell population is compared to the expression level of the same gene in a reference cell population that is not exposed to the test agent.

An agent capable of suppressing the expression of the LY6K gene has marked clinical benefit. Such agents can be further tested for the ability to forestall or prevent lung or esophageal carcinomal growth in animals or test subjects.

In a further embodiment, the present invention provides methods for screening candidate agents which act on the targets in the treatment of LC and/or EC. As discussed in detail above, by controlling the expression level of the LY6K gene or the activity level of its gene product, one can control the onset and progression of LC and/or EC. Thus, candidate agents, which act on the targets in the treatment of LC and/or EC, can be identified through screening methods that use such expression and activity levels as indices of the cancerous or non-cancerous state. In the context of the present invention, such screening can include, for example, the following steps:

(a) contacting a test compound with a polypeptide encoded by a LY6K polynucleotide

(b) detecting the binding activity between the polypeptide and the test compound; and

(c) selecting the test compound that binds to the polypeptide.

Alternatively, the screening methods of the present invention can include the following steps:

(a) contacting a candidate compound with a cell expressing the LY6K gene; and

(b) selecting the candidate compound that reduces the expression level of the LY6K gene, as compared to the expression level detected in the absence of the candidate compound.

Cells expressing the LY6K gene include, but are not limited to, for example, cell lines established from LC or EC; such cells can be used for the above screening of the present invention.

Alternatively, the screening methods of the present invention can include the following steps:

(a) contacting a test compound with a polypeptide encoded by a LY6K polynucleotide;

(b) detecting the biological activity of the polypeptide of step (a); and

(c) selecting a compound that suppresses the biological activity of the polypeptide encoded by the LY6K polynucleotide, as compared to the biological activity detected in the absence of the test compound.

A protein for use in the screening methods of the present invention can be obtained as a recombinant protein using the known nucleotide sequence for the LY6K gene. Based on the information regarding the LY6K gene and its encoded protein, one skilled in the art can select any biological activity of the protein as an index for screening and any suitable measurement method to assay for the selected biological activity. Specifically, the LY6K protein is known to have a cell proliferating activity. Therefore, the biological activity can be determined using such cell proliferating activity.

Alternatively, the screening methods of the present invention can include the following steps:

(a) contacting a candidate compound with a cell into which a vector, containing the transcriptional regulatory region of LY6K genes and a reporter gene that is expressed under the control of the transcriptional regulatory region, has been introduced;

(b) measuring the expression or activity of said reporter gene; and

(c) selecting the candidate compound that reduces the expression or activity level of said reporter gene, as compared to the expression or activity level detected in the absence of the candidate compound.

Suitable reporter genes and host cells are well known in the art. A reporter construct suitable for the screening methods of the present invention can be prepared by using a transcriptional regulatory region of the LY6K gene. A nucleotide segment containing the transcriptional regulatory region can be isolated from a genome library based on the nucleotide sequence information for the LY6K gene.

Selecting a Therapeutic Agent for treating LC and/or EC:

Differences in the genetic makeup of individuals can result in differences in their relative abilities to metabolize various drugs. An agent that is metabolized in a subject to act as an anti-LC and/or EC agent can manifest itself by inducing a change in a gene expression pattern in the subject's cells from that is characteristic of a cancerous state to a gene expression pattern that is characteristic of a non-cancerous state. Accordingly, the differentially expressed LY6K gene allows for a putative therapeutic or prophylactic inhibitor of LC and/or EC to be tested in a test cell population from a selected subject in order to determine if the agent is a suitable inhibitor of LC and/or EC in the subject.

To identify an inhibitor of LC and/or EC that is appropriate for a specific subject, a test cell population from the subject is exposed to a therapeutic agent, and the expression of the LY6K gene is determined.

In the context of the methods of the present invention, the test cell population contains LC and/or EC cells expressing the LY6K gene. Preferably, the test cell population includes epithelial cells. For example, a test cell population can be incubated in the presence of a candidate agent and the pattern of gene expression of the test cell population can be measured and compared to one or more reference expression profiles, e.g., an LC reference expression profile, an EC reference expression profile or normal reference expression profile, e.g., a non-LC and non-EC reference expression profile.

A decrease in the expression of the LY6K gene in a test cell population relative to a reference cell population containing LC and/or EC indicates that the agent has therapeutic utility. Alternatively, a similarity in the expression of the LY6K gene in the test cell population and the reference cell population indicates that the agent has alternate therapeutic utility.

In the context of the present invention, the test agent can be any compound or composition. Exemplary test agents include, but are not limited to, immunomodulatory agents (e.g., antibodies), inhibitory oligonuceotides (e.g., antisense oligonucleodies, short-inhibitory oligonucleotides and ribozymes) and small organic compounds.

Candidate Compounds:

A compound isolated by the screening assays of the present invention may serve as a candidate for the development of drugs that inhibit the expression of the LY6K gene or the activity of the protein encoded by the LY6K gene and can be applied to the treatment or prevention of lung cancer and/or esophageal cancer.

Moreover, compounds in which a part of the structure of the compound inhibiting the activity of protein encoded by the LY6K gene is converted by addition, deletion and/or replacement are also included as the compounds obtainable by the screening methods of the present invention.

When administrating a compound isolated by the methods of the present invention as a pharmaceutical for humans and other mammals, including without limitation, mice, rats, hamsters, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the needs of the patient, the drugs can be taken orally, such as in the form of sugar-coated tablets, capsules, elixirs and microcapsules, or non-orally, such as in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, including gelatin, corn starch, tragacanth gum and arabic gum; excipients, including crystalline cellulose; swelling agents, including corn starch, gelatin and alginic acid; lubricants, including magnesium stearate; sweeteners, including sucrose, lactose or saccharin; and flavoring agents, including peppermint, spearmint, Gaultheria adenothrix oil and cherry. When the unit-dose form is a capsule, a liquid carrier, including an oil, can be further included in the above ingredients. Sterile composites for injection can be formulated following normal drug implementations using vehicles, for example, distilled water or saline solution, suitable for injection.

Physiological saline, glucose, and other isotonic liquids, including adjuvants, such as D-sorbitol, D-mannose, D-mannitol, and sodium chloride, can be used as aqueous solutions for injection. These can be used in conjunction with suitable solubilizers, for example, alcohols including ethanol; polyalcohols, including propylene glycol and polyethylene glycol; and non-ionic surfactants, including Polysorbate 80 (™) and HCO-50.

Sesame oil or soy-bean oil can be used as an oleaginous liquid, can be used in conjunction with benzyl benzoate or benzyl alcohol as a solubilizer, and can be formulated with a buffer, including phosphate buffer and sodium acetate buffer; a pain-killer, including procaine hydrochloride; a stabilizer, including benzyl alcohol and phenol; and/or an anti-oxidant. A prepared injection can be filled into a suitable ampoule.

Methods well known to those skilled in the art can be used to administer the pharmaceutical composition of the present invention to patients, for example as an intraarterial, intravenous, or percutaneous injection or as an intranasal, transbronchial, intramuscular or oral administration If said compound is encodable by a DNA, the DNA can be inserted into a vector for gene therapy and the vector administered to a patient to perform the therapy. In either context, the dosage and method of administration may vary according to the body-weight, age, and symptoms of the patient; however, one skilled in the art can suitably select them.

For example, although the dose of a compound that binds to a protein of the present invention and regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weighing about 60 kg).

When administering the compound parenterally, in the form of an injection to a normal adult human (weighing about 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount can be routinely calculated by converting to 60 kg of body-weight.

Monitoring and Prognosing Lung Cancer and/or Esophageal Cancer

Assessing the Efficacy of Treatment:

The differentially expressed LY6K gene identified herein also allows for the course of treatments for LC and/or EC to be monitored and assessed. Alternatively, according to the present invention, an intermediate result for monitoring the course of treatment of LC and/or EC may be provided. Such intermediate results may be combined with additional information to assist a doctor, nurse, or other practitioner to determine that a subject suffers from lung cancer or esophageal cancer. Thus, LY6K gene or protein encoded thereby is useful prognostic marker for monitoring clinical outcome of LC and/or EC. Alternatively, the present invention may be used to detect cancerous cells in a subject-derived tissue, and provide a doctor with useful information to assess the course of treatment of LC and/or EC. In this method, a test cell population is provided from a subject undergoing treatment for LC and/or EC. If desired, test cell populations are obtained from the subject at various time points, before, during, and/or after treatment. Expression of the LY6K gene in the test cell population is then determined and compared to expression of the same genes in a reference cell population which includes cells whose LC state and/or EC state is known. In the context of the present invention, the reference cells should not have been exposed to the treatment of interest.

In the context of monitoring and assessing a particular course of treatment for LC and/or EC, the biological sample should be derived from a subject undergoing treatment for non-small cell lung cancer and/or esophageal squamous-cell carcinoma. Preferably, multiple test biological samples are obtained from the subject at various time points before, during or after the treatment.

If the reference cell population contains no LC cells and no EC cells, a similarity in the expression of the LY6K gene in the test cell population and the reference cell population indicates that the treatment of interest is efficacious. However, a difference in the expression of the LY6K gene in the test cell population and a normal control reference cell population indicates a less favorable clinical outcome or prognosis. Similarly, if the reference cell population contains LC cells and/or EC cells, a difference between the expression of the LY6K gene in the test cell population and the reference cell population indicates that the treatment of interest is efficacious, while a similarity in the expression of the LY6K gene in the test population and an LC control reference cell population and/or an EC control reference cell population indicates a less favorable clinical outcome or prognosis.

Additionally, the expression level of the LY6K gene determined in a biological sample from a subject obtained after treatment (i.e., post-treatment levels) can be compared to the expression level of the LY6K gene determined in a biological sample from a subject obtained prior to treatment onset (i.e., pre-treatment levels). A decrease in the expression level in a post-treatment sample indicates that the treatment of interest is efficacious while an increase or maintenance in the expression level in the post-treatment sample indicates a less favorable clinical outcome or prognosis.

As used herein, the term “efficacious” indicates that the treatment leads to a reduction in the expression of LY6K gene or a decrease in size, prevalence, or metastatic potential of LC and/or EC in a subject. When a treatment of interest is applied prophylactically, the term “efficacious” means that the treatment retards or prevents a lung cancer and/or an esophageal tumor from forming or retards, prevents, or alleviates a symptom of clinical LC and/or EC. Assessment of lung or esophageal tumors can be made using standard clinical protocols.

In addition, efficaciousness can be determined in association with any known method for diagnosing or treating LC and/or EC. LC and/or EC can be diagnosed, for example, histopathologically or alternatively by identifying symptomatic anomalies, e.g., weight loss, loss of appetite, abdominal pain, back pain, anorexia, nausea, vomiting and generalized malaise, weakness, and jaundice.

Assessing the Prognosis of a Subject with Lung cancer and/or Esophageal Cancer:

The present invention also provides methods for assessing the prognosis of a subject with LC or EC, such methods including the step of comparing the expression of the LY6K gene in a test cell population to the expression of the LY6K gene in a reference cell population from patients over a spectrum of disease stages. By comparing the gene expression of the LY6K gene in the test cell population and the reference cell population(s), or by comparing the pattern of gene expression over time in test cell populations from the subject, the prognosis of the subject can be assessed.

Alternatively, according to the present invention, an intermediate result for assessing the prognosis of a subject with LC or EC may be provided. Such intermediate result may be combined with additional information to assist a doctor, nurse, or other practitioner to determine that a subject suffers from lung cancer or esophageal cancer. Alternatively, the present invention may be used to detect cancerous cells in a subject-derived tissue, and provide a doctor with useful information to assess the prognosis of a subject with LC or EC.

For example, an increase in the expression of the LY6K gene in a test sample as compared to a normal control sample indicates a less favorable prognosis. Conversely, a similarity in the expression of the LY6K gene, in a test sample as compared to normal control sample, indicates a more favorable prognosis for the subject.

Treating and Preventing Lung Cancer and/or Esophageal Cancer

Methods of Inhibiting Lung Cancer and/or Esophageal Cancer:

The present invention further provides a method for preventing, treating and/or alleviating one or more symptoms of LC and/or EC in a subject by decreasing the expression of the LY6K gene (or the activity of its gene product). Suitable therapeutic compounds can be administered prophylactically or therapeutically to a subject suffering from or at risk of (or susceptible to) developing LC and/or EC. Prophylactic administration occurs prior to the manifestation of overt clinical symptoms of disease, such that a disease or disorder is prevented or alternatively delayed in its progression. Such subjects can be identified using standard clinical methods or by detecting an aberrant level of expression of the LY6K gene or aberrant activity of its gene product. In the context of the present invention, suitable therapeutic agents include, for example, inhibitors of cell cycle regulation, cell proliferation.

The therapeutic methods of the present invention can include the step of decreasing the expression, function, or both, of gene product of LY6K genes whose expression is aberrantly increased (“up-regulated” or “over-expressed” gene) in lung cells and/or esophageal cells. Expression can be inhibited in any of several ways known in the art. For example, expression can be inhibited by administering to the subject a compound, e.g., a nucleic acid that inhibits, or antagonizes the expression of the LY6K gene, e.g., an antisense oligonucleotide or small interfering RNA which disrupts expression of the LY6K gene.

Inhibitory Nucleic Acids:

As noted above, inhibitory nucleic acids (e.g., antisense oligonucleotides, siRNA, ribozymes) complementary to the nucleotide sequence of the LY6K gene can be used to reduce the expression level of the gene. For example, inhibitory nucleic acids complementary to the LY6K gene that are up-regulated in lung cancer or esophageal cancer are useful for the treatment of lung cancer or esophageal cancer. Specifically, the inhibitory nucleic acids of the present invention can act by binding to the LY6K gene, or mRNAs corresponding thereto, thereby inhibiting the transcription or translation of the gene, promoting the degradation of the mRNA, and/or inhibiting the expression of protein encoded by the LY6K gene, thereby, inhibiting the function of the protein.

The term “inhibitory nucleic acids” as used herein encompasses both nucleotides that are entirely complementary to the target sequence and those having a mismatch of one or more nucleotides, so long as the inhibitory nucleic acids can specifically hybridize to the target sequences. The inhibitory nucleic acids of the present invention include polynucleotides that have a sequence identity of at least 70%, 75%, 80%, 85%, 90%, 95%, 96%, 97%, 98%, 99% or higher over a span of at least 15 continuous nucleotides. Algorithms known in the art can be used to determine the sequence identity.

One useful algorithm is BLAST 2.0, originally described in Altschul et al., (1990) J. Mol. Biol. 215: 403-10. Software for performing BLAST analyses is publicly available through the National Center for Biotechnology Information (available on the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff (1989) Proc. Natl. Acad. Sci. USA 89: 10915-9).

An additional example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, (1987) J. Mol. Evol. 35: 351-60. The method used is similar to the method described by Higgins & Sharp, (1989) CABIOS 5:151-3. The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison. For example, in order to determine conserved amino acids in a monomer domain family or to compare the sequences of monomer domains in a family, the sequence of the invention, or coding nucleic acids, are aligned to provide structure-function information.

The antisense nucleic acids of the present invention act on cells producing the proteins encoded by EC-associated marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid of the present invention can be made into an external preparation, for example, a liniment or a poultice, by admixing it with a suitable base material which is inactive against the nucleic acid.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids of the present invention can be given to the patient by direct application onto the ailing site or by injection into a blood vessel so that it will reach the site of ailment. An antisense-mounting medium can also be used to increase durability and membrane-permeability. Examples include, but are not limited to, liposomes, poly-L-lysine, lipids, cholesterol, lipofectin or derivatives of these.

The dosage of the inhibitory nucleic acids of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein of the present invention and are thereby useful for suppressing the biological activity of the protein of the invention. In addition, expression-inhibitors, including antisense nucleic acids of the present invention, are useful in that they can inhibit the biological activity of a protein of the present invention.

The methods of the present invention can be used to alter LY6K expression in a cell. Binding of the antisense nucleic acids to a transcript complementary to the LY6K gene in the target cell results in a reduction in the protein production by the cell. The length of the oligonucleotide is at least 10 nucleotides and can be as long as the naturally-occurring transcript. Preferably, the oligonucleotide is less than 75, 50, 25 nucleotides in length. Most preferably, the oligonucleotide is 19-25 nucleotides in length.

The antisense nucleic acids of present invention include modified oligonucleotides. For example, thioated oligonucleotides can be used to confer nuclease resistance to an oligonucleotide.

The term “polynucleotide” and “oligonucleotide” are used interchangeably herein unless otherwise specifically indicated and are referred to by their commonly accepted single-letter codes. The terms apply to nucleic acid (nucleotide) polymers in which one or more nucleic acids are linked by ester bonding. The polynucleotide or oligonucleotide may be composed of DNA, RNA or a combination thereof.

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

Also, an siRNA against the LY6K gene can be used to reduce the expression level of the LY6K gene. Herein, term “siRNA” refers to a double stranded RNA molecule which prevents translation of a target mRNA. Standard techniques for introducing siRNA into the cell can be used, including those in which DNA is a template from which RNA is transcribed. In the context of the present invention, the siRNA is composed of a sense nucleic acid sequence and an anti-sense nucleic acid sequence against the LY6K gene. The siRNA is constructed such that a single transcript has both the sense and complementary antisense sequences from the target gene, e.g., a hairpin. The siRNA may either be a dsRNA or shRNA.

As used herein, the term “dsRNA” refers to a construct of two RNA molecules having sequences complementary to one another annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a LY6K sense nucleic acid sequence (also referred to as “sense strand”), a LY6K antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules having sequences complementary to one another annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, including first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-O-methyl-4′ linked ribonucleotides, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyi, or 7-alkenyi purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule including both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA preferably includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

In another embodiment, halogenated RNAs, RNAs partially replaced with DNAs, or methylated RNAs can be used to confer RNAase resistance to the siRNA. Such nucleic acid derivatives that confer RNAase resistance are also included in the double-stranded RNA. In the present invention, the double stranded molecule may include a double stranded RNA constructed from ribonucleotides, modified ribonucleotides, or ribonucleotide derivatives.

An siRNA of the LY6K gene hybridizes to target mRNA and thereby decreases or inhibits production of the polypeptides encoded by the LY6K gene by associating with the normally single-stranded mRNA transcript, thereby interfering with translation and thus, expression of the protein. In the context of the present invention, an siRNA is preferably less than 500, 200, 100, 50, or 25 nucleotides in length. More preferably an siRNA is 19-25 nucleotides in length. Exemplary nucleic acid sequence for the production of LY6K siRNA includes the sequences of nucleotides of SEQ ID NOs: 11 as the target sequence. In order to enhance the inhibition activity of the siRNA, one or more uridine (“u”) nucleotides can be added to 3′end of the antisense strand of the target sequence. The number of “u's” to be added is at least 2, generally 2 to 10, preferably 2 to 5. The added “u's” form a single strand at the 3′end of the antisense strand of the siRNA.

An siRNA of the LY6K gene can be directly introduced into the cells in a form that is capable of binding to the mRNA transcripts. Alternatively, a DNA encoding the siRNA can be carried in a vector.

Vectors can be produced, for example, by cloning an LY6K gene target sequence into an expression vector having operatively-linked regulatory sequences flanking the sequence in a manner that allows for expression (by transcription of the DNA molecule) of both strands (Lee, N. S., et al., (2002) Nature Biotechnology 20: 500-5). An RNA molecule that is antisense to mRNA of the LY6K gene is transcribed by a first promoter (e.g., a promoter sequence 3′ of the cloned DNA) and an RNA molecule that is the sense strand for the mRNA of the LY6K gene is transcribed by a second promoter (e.g., a promoter sequence 5′ of the cloned DNA). The sense and antisense strands hybridize in vivo to generate siRNA constructs for silencing of the LY6K gene. Alternatively, the two constructs can be utilized to create the sense and anti-sense strands of a siRNA construct. Cloned LY6K gene can encode a construct having secondary structure, e.g., hairpins, wherein a single transcript has both the sense and complementary antisense sequences from the target gene.

A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′,

wherein [A] is a ribonucleotide sequence corresponding to a sequence of the LY6K gene,

[B] is a ribonucleotide sequence composed of 3 to 23 nucleotides, and

[A′] is a ribonucleotide sequence having the complementary sequence of [A].

The region [A] hybridizes to [A′], and then a loop composed of region [B] is formed. The loop sequence can be 3 to 23 nucleotides in length. The loop sequence, for example, can be selected from the following sequences (found on the worldwide web at ambion.com/techlib/tb/tb_{—}506.html). Furthermore, a loop sequence consisting of 23 nucleotides also provides active siRNA (Jacque, J. M., et al., (2002) Nature 418 : 435-8.).

CCC, CCACC or CCACACC: Jacque, J. M, et al., (2002) Nature, Vol. 418: 435-8.

UUCG: Lee, N. S., et al., (2002) Nature Biotechnology 20: 500-5; Fruscoloni, P., et al., (2003) Proc. Natl. Acad. Sci. USA 100(4): 1639-44.

UUCAAGAGA: Dykxhoorn, D. M., et al., (2003) Nature Reviews Molecular Cell Biology 4: 457-67.

Accordingly, in some embodiments, the loop sequence can be selected from group consisting of, CCC, UUCG, CCACC, CCACACC, and UUCAAGAGA. A preferable loop sequence is UUCAAGAGA (“ttcaagaga” in DNA). Exemplary hairpin siRNA suitable for use in the context of the present invention include:

for LY6K-siRNA
AAGGAGGUGCAAAUGGACAGA-[b]-UCUGUCCAUUUGCACCUCCUU
(for target sequence of SEQ ID NO: 11)

The nucleotide sequence of suitable siRNAs can be designed using an siRNA design computer program available from the Ambion website on the worldwide web at ambion.com/techlib/misc/siRNA_finder.html. The computer program selects nucleotide sequences for siRNA synthesis based on the following protocol.

Selection of siRNA Target Sites:

1. Beginning with the AUG start codon of the object transcript, scan downstream for AA dinucleotide sequences. Record the occurrence of each AA and the 3′ adjacent 19 nucleotides as siRNA target sites. Tuschl, et al. Genes Dev 13(24):3191-7(1999) don't recommend against designing siRNA to the 5′ and 3′ untranslated regions (UTRs) and regions near the start codon (within 75 bases) as these may be richer in regulatory protein binding sites. UTR-binding proteins and/or translation initiation complexes can interfere with binding of the siRNA endonuclease complex.

2. Compare the target sites to the human genome database and eliminate from consideration any target sequences with significant sequence identity to other coding sequences. The sequence identity search can be performed using BLAST 2.0 (Altschul S F, et al., Nucleic Acids Res. 1997;25(17):3389-402; Altschul S F, J Mol Biol. 1990;215(3):403-10.), which can be found on the NCBI server at ncbi.nlm.nih.gov/BLAST/.

3. Select qualifying target sequences for synthesis. Using the Ambion algorithm, preferably several target sequences can be selected along the length of the gene to evaluate.

The regulatory sequences flanking the LY6K gene sequences can be identical or different, such that their expression can be modulated independently, or in a temporal or spatial manner. siRNAs are transcribed intracellularly by cloning the LY6K gene templates, respectively, into a vector containing, e.g., a RNA polymerase III transcription unit from the small nuclear RNA (snRNA) U6 or the human H1 RNA promoter. For introducing the vector into the cell, transfection-enhancing agent can be used. FuGENE (Roche diagnostics), Lipofectamine 2000 (Invitrogen), Oligofectamine (Invitrogen), and Nucleofector (Wako pure Chemical) are useful as the transfection-enhancing agent.

The antisense oligonucleotide or siRNA of the present invention inhibits the expression of a polypeptide of the present invention and is thereby useful for suppressing the biological activity of a polypeptide of the invention. Also, expression-inhibitors, including the antisense oligonucleotide or siRNA of the invention, are useful in the point that they can inhibit the biological activity of the polypeptide of the invention. Therefore, a composition composed of one or more antisense oligonucleotides or siRNAs of the present invention is useful for treating an esophageal cancer. Alternatively, the present invention provides use of inhibitory nucleic acids including antisense nucleic acids or siRNAs, or vector expressing the nucleic acids for manufacturing a pharmaceutical composition for treating or preventing a cell proliferative disease, for example cancer, in particular LC and/or EC. Further, the present invention also provides such inhibitory nucleic acids including antisense nucleic acids or siRNAs, or vector expressing the nucleic acids for treating or preventing a cell proliferative disease, for example cancer, in particular LC and/or EC.

Antibodies and Immunotherapy:

Alternatively, the function of the LY6K gene products of the genes over-expressed in LC and EC can be inhibited by administering a compound that binds to or otherwise inhibits the function of the gene products. For example, the compound can be an antibody which binds to the LY6K gene product or gene products.

The present invention refers to the use of antibodies, particularly antibodies against a protein encoded by the LY6K gene, or a fragment of such an antibody. As used herein, the term “antibody” refers to an immunoglobulin molecule having a specific structure, that interacts (i.e., binds) only with the antigen that was used for synthesizing the antibody (i.e., the gene product of an up-regulated marker) or with an antigen closely related thereto. Furthermore, an antibody can be a fragment of an antibody or a modified antibody, so long as it binds to one or more of the proteins encoded by the marker genes. For instance, the antibody fragment can be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-83 (1988)). More specifically, an antibody fragment can be generated by treating an antibody with an enzyme, including papain or pepsin. Alternatively, a gene encoding the antibody fragment can be constructed, inserted into an expression vector, and expressed in an appropriate host cell (see, for example, Co M. S. et al. J. Immunol. 152:2968-76 (1994); Better M. and Horwitz A. H. Methods Enzymol. 178:476-96 (1989); Pluckthun A. and Skerra A. Methods Enzymol. 178:497-515 (1989); Lamoyi E. Methods Enzymol. 121:652-63 (1986); Rousseaux J. et al. Methods Enzymol. 121:663-9 (1986); Bird R. E. and Walker B. W. Trends Biotechnol. 9:132-7 (1991)).

An antibody can be modified by conjugation with a variety of molecules, including polyethylene glycol (PEG). The present invention provides such modified antibodies. The modified antibody can be obtained by chemically modifying an antibody. Such modification methods are conventional in the field.

An antibody of present invention can be bound to a pharmaceutical agent, wherein the antibody is specific for cancer cells. The pharmaceutical agent intensively acts on the cancer cells, therefore, even agents with strong side effects can be used with less side effects, in addition to pharmaceutical agents, there are also reports of approaches where precursors of pharmaceutical agents, enzymes which metabolize the precursors to an active form, and so on are bound to the antibodies. In an alternate embodiment, an antibody of the present invention may be fused, conjugated, or operably linked to a radioisotope to form a radioconjugate. A variety of radioactive isotopes are available for the production of radioconjugate antibodies. Examples include, but are not limited to, At²¹¹, 1¹³¹, 1¹²⁵, Y⁹⁰, Re¹⁸⁶, Re¹⁸⁸, Sm¹⁵³, Bi²¹², P³², and radioactive isotopes of Lu.

Alternatively, an antibody can take the form of a chimeric antibody having a variable region from a nonhuman antibody and a constant region from a human antibody, or a humanized antibody, having a complementarity determining region (CDR) from a nonhuman antibody, a frame work region (FR) and a constant region from a human antibody. Such antibodies can be prepared by using known technologies. Furthermore, in the present invention, an antibody may be a human antibody. For instance, a human antibody may be selected by screening from phage display library. Method for constructing the phage display library and a screening procedure of such antibodies are also well known.

Furthermore, an antibody which has an ADCC or CDC activity and binds especially to cancer cells, can be used for treatment of cancer. Antibody-dependent cell-mediated cytotoxicity and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95: 652-656 (1998). Human effector cells are leukocytes which express one or more FcRs and perform effector functions. Preferably, the cells express at least FcyRI-III and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. Complement dependent cytotoxicity (CDC) refer to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay, e.g., as described in Gazzano-Santoro et al. J. Immunol. Methods 202: 163 (1996), may be performed.

Other compounds have been developed that target and bind to targets in a manner similar to antibodies. Certain of these “antibody mimics” use non-immunoglobulin protein scaffolds as alternative protein frameworks for the variable regions of antibodies. Thus, the term “antibody mimic” refers to non-antibody binding proteins that use non-immunoglobulin protein scaffolds, including adnectins, avimers, single chain polypeptide binding molecules, and antibody-like binding peptidomimetics, as discussed in more detail below. One of skill will recognize that any method of using antibodies described in this document could also be carried out using antibody mimics.

Ku et al. (Proc. Natl. Acad. Sci. U.S.A. 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. U.S.A. 96(5):1898-1903 (1999)) discloses an antibody mimic based on a lipocalin scaffold (Anticalin®). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies.

Anticalins® are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. Mol. Biol. 49(2):209-216 (2003)) discusses a methodology for reducing antibodies into smaller peptidomimetics, they term “antibody like binding peptidomemetics” (ABiP) which can also be useful as an alternative to antibodies.

Silverman et al. (Nat. Biotechnol. (2005), 23: 1556-1561) discloses fusion proteins that are single-chain polypeptides composed of multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties are also mimicked by compounds composed of RNA molecules and unnatural oligomers (e.g., protease inhibitors, benzodiazepines, purine derivatives and beta-turn mimics) all of which are suitable for use with the present invention.

Cancer therapies directed at specific molecular alterations that occur in cancer cells have been validated through clinical development and regulatory approval of anticancer drugs, including, for example, trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F and Tortora G. Clin Cancer Res. 2001;7(10):2958-70. Review; Slamon D J, et al., N Engl J Med. 2001;344(11):783-92; Rehwald U, et al., Blood. 2003;101(2):420-4; Fang G, et al., (2000). Blood, 96, 2246-53.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56; Klejman A, et al., (2002). Oncogene, 21, 5868-76.). Therefore, future cancer treatments will likely involve a combination of conventional drugs with target-specific agents aimed at different characteristics of tumor cells, for example, angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid molecules as therapy to counteract aberrant expression of the differentially expressed genes or aberrant activity of their gene products.

Diseases and disorders that are characterized by increased (relative to a subject not suffering from the disease or disorder) expression levels or biological activities of genes and gene products, respectively, can be treated with therapeutics that antagonize (i.e., reduce or inhibit) activity of the over-expressed gene or genes. Therapeutics that antagonize activity can be administered therapeutically or prophylactically.

Accordingly, therapeutics that can be utilized in the context of the present invention include, e.g., (i) a polypeptide of the LY6K gene, or analogs, derivatives, fragments or homologs thereof; (ii) antibodies to the LY6K gene or gene product; (iii) nucleic acids encoding the LY6K gene; (iv) antisense nucleic acids or nucleic acids that are “dysfunctional” (i.e., due to a heterologous insertion within the nucleic acids of the LY6K gene); (v) small interfering RNA (siRNA); or (vi) modulators (i.e., inhibitors, agonists and antagonists that alter the interaction between LY6K polypeptide and its binding partner). The dysfunctional antisense molecules are utilized to “knockout” endogenous function of a polypeptide by homologous recombination (see, e.g., Capecchi, Science 244: 1288-92 1989).

Increased or decreased levels can be readily detected by quantifying peptide and/or RNA of LY6K, by obtaining a patient tissue sample (e.g., from biopsy tissue) and assaying it in vitro for RNA of LY6K or peptide levels, structure and/or activity of the LY6K peptides. Methods that are well known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease or disorder, such that a disease or disorder is prevented or, alternatively, delayed in its progression.

Therapeutic methods of the present invention can include the step of contacting a cell with an agent that modulates one or more of the activities of the gene products of the LY6K gene. Examples of agents that modulates protein activity include, but are not limited to, nucleic acids, proteins, naturally occurring cognate ligands of such proteins, peptides, peptidomimetics, and other small molecule.

Vaccinating Against Lung Cancer and Esophageal Cancer:

The present invention also relates to methods of treating or preventing lung cancer and esophageal cancer in a subject including the step of administering to said subject a vaccine containing one or more polypeptides encoded by LY6K nucleic acid, an immunologically active fragment of said polypeptide (i.e., an epitope), or a polynucleotide encoding such a polypeptide or fragment thereof. Examples of LY6K (URLC10) derived peptide vaccines for treating cancer are described in the WIPO Publication, WO 2006/90810, the entire contents of which are incorporated by reference herein.

Administration of the polypeptide induces an anti-tumor immunity in a subject. To induce anti-tumor immunity, one or more polypeptides encoded by LY6K nucleic acids, an immunologically active fragment(s) of said polypeptides, or polynucleotide(s) encoding such polypeptide(s) or fragment(s) thereof is administered to subject in need thereof. Furthermore, the one or more polypeptides encoded by the LY6K nucleic acids can induce anti-tumor immunity against metastatic and recurrent lung cancer or esophageal cancer, respectively. The polypeptide or the immunologically active fragments thereof are useful as vaccines against LC or EC. In some cases, the proteins or fragments thereof can be administered in a form bound to the T cell receptor (TCR) or presented by an antigen presenting cell (APC), including macrophages, dendritic cells (DC), or B-cells. Due to the strong antigen presenting ability of DC, the use of DC is most preferable among the APCs.

Identification of immunologically active fragments (i.e., epitopes) is well known in the art. B-cell epitopes can be formed both from contiguous amino acids or non-contiguous amino acids juxtaposed by tertiary folding of a protein. Epitopes formed from contiguous amino acids are typically retained on exposure to denaturing solvents whereas epitopes formed by tertiary folding (i.e., conformationally determined) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen (e.g., a competitive ELISA or solid phase radioimmunoassay (SPRIA)). T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis. 170, 1110-19 (1994)), by antigen-dependent killing (cytotoxic T lymphocyte assay, Tigges et al., J. Immunol. (1996) 156:3901-10) or by cytokine secretion. Methods for determining immunogenic epitopes are described, for example, in Reineke, et al., Curr Top Microbiol Immunol (1999) 243:23-36; Mahler, et al., Clin Immunol (2003) 107:65-79; Anthony and Lehmann, Methods (2003) 29:260-9; Parker and Tomer, Methods Mol Biol (2000) 146:185-201; DeLisser, Methods Mol Biol (1999) 96:11-20; Van de Water, et al., Clin Immunol Immunopathol (1997) 85:229-35; Carter, Methods Mol Biol (1994) 36:207-23; and Pettersson, Mol Biol Rep (1992) 16:149-53.

In the present invention, a vaccine against LC and/or EC refers to a substance that has the ability to induce anti-tumor immunity upon inoculation into animals. According to the present invention, polypeptides encoded by the LY6K gene, or fragments thereof, are HLA-A24 or HLA-A*0201 restricted epitopes peptides that induce potent and specific immune response against LC and/or EC cells expressing the LY6K gene. Thus, the present invention also encompasses methods of inducing anti-tumor immunity using the polypeptides. In general, anti-tumor immunity includes immune responses including as follows:

induction of cytotoxic lymphocytes against tumors,

induction of antibodies that recognize tumors, and

induction of anti-tumor cytokine production.

Therefore, when a certain protein induces any one of these immune responses upon inoculation into an animal, the protein is determined to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. Specifically, a foreign substance that enters the living body is presented to T cells and B cells by the action of antigen presenting cells (APCs). T cells that respond to the antigen presented by the APCs in an antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell via an APC, and detecting the induction of CTLs. Furthermore, APCs have the effect of activating CD4+ T cells, CD8+ T cells, macrophages, eosinophils, and NK cells. Since CD4+ T cells and CD8+ T cells are also important in anti-tumor immunity, the anti-tumor immunity-inducing action of the peptide can be evaluated using the activation effect of these cells as indicators. See, Coligan, Current Protocols in Immunology, supra.

A method for evaluating the inducing action of CTLs using dendritic cells (DCs) as the APC is well known in the art. DCs are a representative APCs having the strongest CTL-inducing action among APCs. In this method, the test polypeptide is initially contacted with DCs, and then the DCs are contacted with T cells. Detection of T cells having cytotoxic effects against the cells of interest after the contact with DC shows that the test polypeptide has an activity of inducing the cytotoxic T cells. Activity of CTLs against tumors can be detected, for example, using the lysis of ⁵¹Cr-labeled tumor cells as the indicator. Alternatively, methods of evaluating the degree of tumor cell damage using ³H-thymidine uptake activity or LDH (lactose dehydrogenase)-release as the indicator is also well known.

Apart from DCs, peripheral blood mononuclear cells (PBMCs) can also be used as the APC. The induction of CTLs has been reported to be enhanced by culturing PBMCs in the presence of GM-CSF and IL-4. Similarly, CTLs have been shown to be induced by culturing PBMCs in the presence of keyhole limpet hemocyanin (KLH) and IL-7.

Test polypeptides confirmed to possess CTL-inducing activity by these methods are deemed to be polypeptides having DC activation effect and subsequent CTL-inducing activity. Therefore, polypeptides that induce CTLs against tumor cells are useful as vaccines against tumors. Furthermore, APCs that have acquired the ability to induce CTLs against tumors through contact with the polypeptides are also useful as vaccines against tumors. Furthermore, CTLs that have acquired cytotoxicity due to presentation of the polypeptide antigens by APCs can be also be used as vaccines against tumors. Such therapeutic methods for tumors, using anti-tumor immunity due to APCs and CTLs, are referred to as cellular immunotherapy.

Generally, when using a polypeptide for cellular immunotherapy, efficiency of the

CTL-induction is known to be increased by combining a plurality of polypeptides having different structures and contacting them with DCs. Therefore, when stimulating DCs with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

Anti-tumor immunity is induced by administering the vaccine of this invention, and the induction of anti-tumor immunity enables treatment and prevention of LC and/or EC. Therapy against cancer or prevention of the onset of cancer includes any of the following steps, including inhibition of the growth of cancerous cells, involution of cancer, and suppression of the occurrence of cancer. A decrease in mortality and morbidity of individuals having cancer, decrease in the levels of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA can be used for statistical analysis.

The above-mentioned protein having immunological activity or a vector encoding the protein can be combined with an adjuvant. An adjuvant refers to a compound that enhances the immune response against the protein when administered together (or successively) with the protein having immunological activity. Exemplary adjuvants include, but are not limited to, cholera toxin, salmonella toxin, alum, and such, but are not limited thereto. Furthermore, the vaccine of this invention can be combined appropriately with a pharmaceutically acceptable carrier. Examples of such carriers include sterilized water, physiological saline, phosphate buffer, culture fluid, and such. Furthermore, the vaccine can contain as necessary, stabilizers, suspensions, preservatives, surfactants, and such. The vaccine can be administered systemically or locally, for example, through intradermal, intramuscular, subcutaneous, transdermal, buccal, or intranasal routes. Vaccine administration can be performed by single administration, or boosted by multiple administrations. Doses are as set forth below.

When using an APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APCs or CTLs, the cells can be administered to the subject. APCs can be also induced by introducing a vector encoding the polypeptide into PBMCs ex vivo. APCs or CTLs induced in vitro can be cloned prior to administration. By cloning and growing cells having high activity of damaging target cells, cellular immunotherapy can be performed more effectively. Furthermore, APCs and CTLs isolated in this manner can be used for cellular immunotherapy not only against individuals from whom the cells are retrieved, but also against similar types of tumors from other individuals.

General methods for developing vaccines are described, for example, in Vaccine Protocols, Robinson and Cranage, Eds., 2003, Humana Press; Marshall, Vaccine Handbook: A Practical Guide for Clinicians, 2003, Lippincott Williams & Wilkins; and Vaccine Delivery Strategies, Dietrich, et al., Eds., 2003, Springer Verlag.

Pharmaceutical Compositions:

Furthermore, a pharmaceutical composition for treating or preventing a cell proliferative disease, for example cancer, in particular LC and/or EC, containing a pharmaceutically effective amount of the polypeptide of the present invention is provided. The pharmaceutical composition can be used for raising anti tumor immunity. Alternatively, the present invention provides use of LY6K protein or gene encoding the protein for manufacturing a pharmaceutical composition for treating or preventing a cell proliferative disease, for example cancer, in particular LC and/or EC. Further, the present invention also provides LY6K protein or gene encoding the protein for treating or preventing a cell proliferative disease, for example cancer, in particular LC and/or EC.

In the context of the present invention, suitable pharmaceutical formulations include, but are not limited to, those suitable for oral, rectal, nasal, topical (including buccal and sub-lingual), vaginal or parenteral (including intramuscular, subcutaneous and intravenous) administration, or for administration by inhalation or insufflation. Preferably, administration is intravenous. The formulations are optionally packaged in discrete dosage units.

Pharmaceutical formulations suitable for oral administration include, but are not limited to, capsules, cachets or tablets, each containing a predetermined amount of active ingredient. Suitable formulations also include, but are not limited to, powders, granules, solutions, suspensions and emulsions. The active ingredient is optionally administered as a bolus electuary or paste. Tablets and capsules for oral administration can contain conventional excipients, including, but not limited to, binding agents, fillers, lubricants, disintegrant and/or wetting agents. A tablet can be made by compression or molding, optionally with one or more formulational ingredients. Compressed tablets can be prepared by compressing in a suitable machine the active ingredients in a free-flowing form, for example, a powder or granules, optionally mixed with a binder, lubricant, inert diluent, lubricating, surface active and/or dispersing agent. Molded tablets can be made by molding in a suitable machine a mixture of the powdered compound moistened with an inert liquid diluent. The tablets can be coated according to methods well known in the art. Oral fluid preparations can be in the form of, for example, aqueous or oily suspensions, solutions, emulsions, syrups or elixirs, or can be presented as a dry product for constitution with water or other suitable vehicle before use. Such liquid preparations can contain conventional additives, for example, suspending agents, emulsifying agents, non-aqueous vehicles (which can include edible oils), and/or preservatives. The tablets can optionally be formulated so as to provide slow or controlled release of the active ingredient therein. A package of tablets can contain one tablet to be taken on each of the month.

Formulations suitable for parenteral administration include aqueous and non-aqueous sterile injection solutions, optionally contain anti-oxidants, buffers, bacteriostats and solutes which render the formulation isotonic with the blood of the intended recipient; as well as aqueous and non-aqueous sterile suspensions including suspending agents and/or thickening agents. The formulations can be presented in unit dose or multi-dose containers, for example as sealed ampoules and vials, and can be stored in a freeze-dried (lyophilized) condition, requiring only the addition of the sterile liquid carrier, for example, saline, water-for-injection, immediately prior to use. Alternatively, the formulations can be presented for continuous infusion. Extemporaneous injection solutions and suspensions can be prepared from sterile powders, granules and tablets of the kind previously described.

Formulations suitable for rectal administration include suppositories with standard carriers for example, cocoa butter or polyethylene glycol. Formulations suitable for topical administration in the mouth, for example, buccally or sublingually, include lozenges, containing the active ingredient in a flavored base, for example, sucrose and acacia or tragacanth, and pastilles, containing the active ingredient in a base, for example, gelatin and glycerin or sucrose and acacia. For intra-nasal administration, the compounds of the invention can be used as a liquid spray, a dispersible powder, or in the form of drops. Drops can be formulated with an aqueous or non-aqueous base also including one or more dispersing agents, solubilizing agents and/or suspending agents.

For administration by inhalation the compounds can be conveniently delivered from an insufflator, nebulizer, pressurized packs or other convenient means of delivering an aerosol spray. Pressurized packs can include a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount.

Alternatively, for administration by inhalation or insufflation, the compounds can take the form of a dry powder composition, for example a powder mix of the compound and a suitable powder base, for example, lactose or starch. The powder composition can be presented in unit dosage form, for example, as capsules, cartridges, gelatin or blister packs, from which the powder can be administered with the aid of an inhalator or insufflators.

Other formulations include implantable devices and adhesive patches which release a therapeutic agent.

When desired, the above described formulations, adapted to give sustained release of the active ingredient, can be employed. The pharmaceutical compositions can also contain other active ingredients, including antimicrobial agents, immunosuppressants and/or preservatives.

It should be understood that in addition to the ingredients particularly mentioned above, the formulations of this invention can include other agents conventional in the art with regard to the type of formulation in question. For example, formulations suitable for oral administration can include flavoring agents.

Preferred unit dosage formulations contain an effective dose, as recited below, or an appropriate fraction thereof, of the active ingredient.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units can conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration can vary depending upon the condition and its severity. In any event, appropriate and optimum dosages can be routinely calculated by those skilled in the art, taking into consideration the above-mentioned factors.

Cancer Diagnosis:

By measuring the level of LY6K in a subject-derived biological sample, the occurrence of cancer or a predisposition to develop cancer in a subject can be determined. Preferably, cancer is either of esophageal and lung cancer, or both. Accordingly, the present invention involves determining (e.g., measuring) the level of LY6K in a biological sample.

By measuring the level of LY6K in subject-derived blood samples, the occurrence of lung cancer or esophageal cancer, or a predisposition to develop lung cancer or esophageal cancer in a subject can be determined. Alternatively, according to the present invention, an intermediate result for examining the condition of a subject may be provided. Such intermediate result may be combined with additional information to assist a doctor, nurse, or other practitioner to determine that a subject suffers from lung cancer or esophageal cancer. Further, the present invention relates to a method for screening a person who is required to be further diagnosed for lung cancer or esophageal cancer. After the screening, persons indicating positive result are recommended to be submitted further screening test, or medical treatment to confirm whether they truly suffer from lung cancer or esophageal cancer. Accordingly, the present invention also provides LY6K protein as blood tumor marker for diagnosing or screening of either of esophageal and lung cancer, or both.

Alternatively, the present invention may be used to detect cancerous cells in a subject-derived tissue, and provide a doctor with useful information to determine that the subject suffers from lung cancer or esophageal cancer. Accordingly, the present invention involves determining (e.g., measuring) the level of LY6K in subject derived samples, such as blood samples. In the present invention, a method for diagnosing lung cancer or esophageal cancer also includes a method for testing or detecting lung cancer or esophageal cancer. Alternatively, in the present invention, diagnosing lung cancer or esophageal cancer also refers to showing a suspicion, risk, or possibility of lung cancer or esophageal cancer in a subject.

Any blood samples may be used for determining the level of LY6K so long as either the LY6K gene or the LY6K protein can be detected in the samples. Preferably, the blood samples includes whole blood, serum, and plasma.

In the present invention, the “level of LY6K in blood samples” refers to the concentration of LY6K present in the blood after correcting the corpuscular volume in the whole blood. One of skill will recognize that the percentage of corpuscular volume in the blood varies greatly between individuals. For example, the percentage of erythrocytes in the whole blood is very different between men and women. Furthermore, differences between individuals cannot be ignored. Therefore, the apparent concentration of a substance in the whole blood which includes corpuscular components varies greatly depending on the percentage of corpuscular volume. For example, even if the concentration in the serum is the same, the measured value for a sample with a large amount of corpuscular component will be lower than the value for a sample with a small amount of corpuscular component. Therefore, to compare the measured values of components in the blood, values for which the corpuscular volume has been corrected are usually used.

For example, by measuring components in the blood using, as samples, serum or plasma obtained by separating blood cells from the whole blood, measured values from which the effect from the corpuscular volume has been removed can be obtained. Therefore, the level of LY6K in the present invention can usually be determined as a concentration in the serum or plasma. Alternatively, it may first be measured as a concentration in the whole blood, then the effect from the corpuscular volume may be corrected. Methods for measuring a corpuscular volume in a whole blood sample are known.

Subjects diagnosed with lung cancer or esophageal cancer according to the present methods are preferably mammals and include humans, non-human primates, mice, rats, dogs, cats, horses and cows. A preferable subject of the present invention is a human. In the present invention, a subject may be a healthy individual or a patient suspected of having either of lung cancer and esophagus cancer, or both. The patient may be diagnosed by the present invention to facilitate clinical decision-making. In another embodiment, the present invention may also be applied to healthy individuals for screening of either of lung cancer and esophagus cancer, or both.

In one embodiment of the present invention, the level of LY6K is determined by measuring the quantity or concentration of LY6K protein in blood samples. Methods for determining the quantity of the LY6K protein in blood samples include immunoassay methods.

In the diagnostic methods of the present invention, the blood concentration of CEA or CYFRA 21-1 or both may be determined, in addition to the blood concentration of LY6K, to detect lung cancer and/or esophageal cancer. Therefore, the present invention provides methods for diagnosing either or both of lung cancer and esophageal cancer, in which the cancers are detected when either the blood concentration of LY6K or the blood concentration of CYFRA 21-1, or both of them, are higher as compared with healthy individuals. Similarly, the prevent invention provides methods for diagnosing either or both of lung cancer and esophageal cancer, in which the cancers are detected when either the blood concentration of LY6K or the blood concentration of CEA, or both of them, are higher as compared with healthy individuals. Alternatively, either or both of lung cancer and esophageal cancer is detected when at least one of blood concentration of LY6K, CYFRA 21-1, and CEA is higher as compared with healthy individuals.

CEA is associated with tumors and the developing fetus. Although CEA was first identified in colon cancer, elevated CEA levels have been found in a variety of cancers apart from colonic, including pancreatic, gastric, lung, and breast cancers. The best use of CEA is as a tumor marker for cancers of the gastrointestinal tract. CYFRA 21-1 measures soluble cytokeratin-19 fragments in serum, and is a useful marker for lung carcinoma, especially squamous cell carcinoma. CYFRA 21-1 is a unique tumor marker that uses two different monoclonal antibodies which recognize the divergent epitope on the N- or C-terminal region of domain 2 of cytokeratin 19 fragment, respectively.

In the present invention, a novel serological marker for lung cancer or esophageal cancer, LY6K, is provided. Improvement in the sensitivity of diagnostic or detection methods for lung cancer or esophageal cancer may be achieved by the present invention. Namely, the present invention provides a method for diagnosing lung cancer or esophageal cancer in a subject, including the steps of:

(a) collecting a blood sample from a subject to be diagnosed;

(b) determining a level of LY6K in the blood sample;

(c) comparing the LY6K level determined in step (b) with that of a normal control wherein a high LY6K level in the blood sample, compared to the normal control, indicates that the subject suffers from lung cancer or esophageal cancer.

In preferable embodiments, the diagnostic or detection method of the present invention may further include the steps of:

(e) determining a level of either or both of CEA and CYFRA21-1 in the blood sample;

(f) comparing the either or both of CEA and CYFRA21-1 level determined in step (e) with that of a normal control; and

(g) judging that high levels of LY6K and either or both of CEA and CYFRA21-1 in the blood sample, compared with the normal control, indicate that the subject suffers from lung cancer and/or esophageal cancer.

Furthermore, method of the measuring targets includes the combination of LY6K and other cancer-associated proteins in biological sample from subject, e.g., CEA and CYFRA21-1. A high level of LY6K expression was associated with poor prognosis of patients with NSCLC (P=0.0026) as well as ESCC (P=0.0455), and multivariate analysis confirmed its independent prognostic value for NSCLC (P=0.0201). The proportion of the serum LY6K-positive cases was 33.9% of NSCLC and 32.1% of ESCC, while only 4.1% of healthy volunteers were falsely diagnosed as positive. The proportion of the serum CEA-positive case was 39.8% of NSCLC, and the proportion of serum CYFRA 21-1-positive case was 39.8 of NSCLC. On the other hand, a combined assay using both LY6K and carcinoembryonic antigen (CEA) judged 64.7% of the lung adenocarcinoma patients as positive while 9.5% of healthy volunteers were falsely diagnosed. The use of both LY6K and CYFRA 21-1 increased sensitivity to detect lung squamous-cell carcinomas up to 70.4%, while false positive rate were only 6.8%. The sensitivity for detection of lung cancer or esophageal cancer may be significantly improved by combining LY6K with CEA and/or CYFRA 21-1. In the preferable embodiments, a patient with positive results of LY6K with CEA and/or CYFRA 21-1 with may be judged to have a high risk of lung cancer or esophageal cancer. The use of a combination of LY6K with CEA and/or CYFRA21-1 as a serological marker for lung cancer and esophageal cancer is novel.

Accordingly, the present invention provides for great improvements in the sensitivity of assays for detecting lung cancer or esophageal cancer in patients, as compared to determinations based on results of measuring CEA or CYFRA 21-1 alone. While not wishing to be bound by theory, it is believed that the fact that the group of CEA-positive or CYFRA 21-1-positive patients and the group of LY6K-positive patients do not match completely is behind this marked improvement. This fact is further described specifically below.

First, among patients who, as a result of CEA or CYFRA 21-1 measurements, were determined to have a lower value than a standard value (i.e. not to have lung cancer or esophageal cancer), there is actually a certain percentage of patients that have lung cancer or esophageal cancer. Such patients are referred to as CEA- or CYFRA 21-1-false negative patients. By combining a determination based on CEA or CYFRA 21-1 with a determination based on LY6K, patients whose LY6K value is above the standard value can be found from among the CEA- or CYFRA 21-1-false negative patients. That is, from among patients falsely determined to be “negative” due to a low blood concentration of CEA or CYFRA 21-1, the present invention provides a means to identify patients actually having lung cancer or esophageal cancer. The sensitivity for detecting lung cancer or esophageal cancer patients is thus improved by the present invention. Generally, simply combining the results from determinations using multiple markers may increase the detection sensitivity, but on the other hand, it often causes a decrease in specificity. However, by determining the best balance between sensitivity and specificity, the present invention has determined a characteristic combination that can increase the detection sensitivity without compromising the specificity.

In the context of the present invention, in order to consider the results of CEA and

CYFRA 21-1 measurements at the same time, for example, the blood concentration of CEA or CYFRA 21-1 may be measured and compared with standard values, in the same way as for the aforementioned comparison between the measured values and standard values of LY6K. For example, how to measure the blood concentration of CEA or CYFRA 21-1 and compare it to standard values are already known. Moreover, ELISA kits for CEA and CYFRA 21-1 are also commercially available. These methods described in known reports can be used in the method of the present invention for diagnosing or detecting lung cancer or esophageal cancer.

In the context of the present invention, the standard value of the blood concentration of LY6K can be determined statistically. For example, the blood concentration of LY6K in healthy individuals can be measured to determine the standard blood concentration of LY6K statistically. When a statistically sufficient population is gathered, a value in the range of twice or three times the standard deviation (S.D.) from the mean value is often used as the standard value. Therefore, values corresponding to the mean value+2×S.D. or mean value+3×S.D. may be used as standard values. The standard values set as described theoretically include 90% and 99.7% of healthy individuals, respectively.

Alternatively, standard values can also be set based on the actual blood concentration of the LY6K protein in lung cancer or esophageal cancer patients. Generally, standard values set this way minimize the percentage of false positives, and are selected from a range of values satisfying conditions that can maximize detection sensitivity. Herein, the percentage of false positives refers to a percentage, among healthy individuals, of patients whose blood concentration of LY6K is judged to be higher than a standard value. On the contrary, the percentage, among healthy individuals, of patients whose blood concentration of LY6K is judged to be lower than a standard value indicates specificity. That is, the sum of the false positive percentage and the specificity is always 1. The detection sensitivity refers to the percentage of patients whose blood concentration of LY6K is judged to be higher than a standard value, among all lung cancer or esophageal cancer patients within a population of individuals for whom the presence of lung cancer or esophageal cancer has been determined.

Furthermore, in the context of the present invention, the percentage of lung cancer or esophageal cancer patients among patients whose LY6K concentration was judged to be higher than a standard value represents the positive predictive value. On the other hand, the percentage of healthy individuals among patients whose LY6K concentration was judged to be lower than a standard value represents the negative predictive value. The relationship between these values is summarized in Table 1 as below. As the relationship shown below indicates, each of the values for sensitivity, specificity, positive predictive value, and negative predictive value, which are indexes for evaluating the diagnostic accuracy for lung cancer or esophageal cancer, varies depending on the standard value for judging the level of the blood concentration of LY6K.

TABLE 1

| Blood | Lung cancer or | | |
|---|---|---|---|
| concentration | esophageal cancer | Healthy | |
| of LY6K | patients | individuals | |
| High | a: True positive | b: False positive | Positive |
| | | | predictive value |
| | | | a/(a + b) |
| Low | c: False negative | d: True negative | Negative |
| | | | predictive value |
| | | | d/(c + d) |
| | Sensitivity | Specificity | |
| | a/(a + c) | d/(b + d) | |

As mentioned previously, a standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a “low false positive ratio”. Considering this situation, for example, values that give the following predicted results may be selected as the preferable standard values in the present invention.

Standard values for which the false positive ratio is 50% or less (that is, standard values for which the specificity is not less than 50%).

Standard values for which the sensitivity is not less than 20%.

In the present invention, the standard values can be set using a receiver operating characteristic (ROC) curve. A ROC curve is a graph that shows the detection sensitivity on the vertical axis and the false positive ratio (that is, “1—specificity”) on the horizontal axis. In the present invention, an ROC curve can be obtained by plotting the changes in the sensitivity and the false positive ratio, which were obtained after continuously varying the standard value for determining the high/low degree of the blood concentration of LY6K.

The “standard value” for obtaining the ROC curve is a value temporarily used for the statistical analyses. The “standard value” for obtaining the ROC curve can generally be continuously varied within a range that allows to cover all selectable standard values. For example, the standard value can be varied between the smallest and largest measured LY6K values in an analyzed population.

Based on the obtained ROC curve, a preferable standard value to be used in the present invention can be selected from a range that satisfies the above-mentioned conditions. Alternatively, a standard value can be selected based on an ROC curve produced by varying the standard values from a range that includes most of the measured LY6K values.

LY6K in the blood can be measured by any conventional method suitable for quantitating proteins. For example, immunoassay, liquid chromatography, surface plasmon resonance (SPR), mass spectrometry, or the like can be used in the context of the present invention. In mass spectrometry, proteins can be quantitated by using a suitable internal standard. For example, isotope-labeled LY6K can be used as the internal standard. The concentration of LY6K in the blood can be determined from the peak intensity of LY6K in the blood and that of the internal standard. Generally, the matrix-assisted laser desorption/ionization (MALDI) method is used for mass spectrometry of proteins. With an analysis method that uses mass spectrometry or liquid chromatography, LY6K can also be analyzed simultaneously with other tumor markers (e.g., CEA or CYFRA 21-1).

A preferable method for measuring LY6K in the context of the present invention is the immunoassay. The amino acid sequence of LY6K is known (Genbank Accession Number HSJ001348, NM_{—}017527). The amino acid sequence of LY6K is shown in SEQ ID NO: 2, and the nucleotide sequence of the cDNA encoding it is shown in SEQ ID NO: 1. Therefore, those skilled in the art can prepare antibodies by synthesizing necessary immunogens based on the amino acid sequence of LY6K. The peptide used as immunogen can be easily synthesized using a peptide synthesizer. The synthetic peptide can be used as an immunogen by linking it to a carrier protein.

Keyhole limpet hemocyanin, myoglobin, albumin, and the like can be used as the carrier protein. Preferable carrier proteins are KLH, bovine serum albumin, and such. The maleimidobenzoyl-N-hydrosuccinimide ester method (hereinafter abbreviated as the MB S method) and the like are generally used to link synthetic peptides to carrier proteins.

Specifically, a cysteine is introduced into the synthetic peptide and the peptide is crosslinked to KLH by MBS using the cysteine's SH group. The cysteine residue may be introduced at the N-terminus or C-terminus of the synthesized peptide.

Alternatively, LY6K can be prepared using the nucleotide sequence of LY6K (Genbank Accession Number HSJ001348, NM_{—}017527), or a portion thereof. DNAs having the necessary nucleotide sequence can be cloned using mRNAs prepared from LY6K-expressing tissues. Alternatively, commercially available cDNA libraries can be used as the cloning source. The obtained genetic recombinants of LY6K, or fragments thereof, can also be used as the immunogen. LY6K recombinants expressed in this manner are preferable as the immunogen for obtaining the antibodies used in the present invention.

Immunogens obtained in this manner are mixed with a suitable adjuvant and used to immunize animals. Known adjuvants include Freund's complete adjuvant (FCA) and incomplete adjuvant. The immunization procedure is repeated at appropriate intervals until an increase in the antibody titer is confirmed. There are no particular limitations on the immunized animals in the present invention. Specifically, animals commonly used for immunization such as mice, rats, or rabbits can be used.

When obtaining the antibodies as monoclonal antibodies, animals that are advantageous for their production may be used. For example, in mice, many myeloma cell lines for cell fusion are known, and techniques for establishing hybridomas with a high probability are already well known. Therefore, mice are a desirable immunized animal to obtain monoclonal antibodies.

Furthermore, the immunization treatments are not limited to in vitro treatments. Methods for immunologically sensitizing cultured immunocompetent cells in vitro can also be employed. Antibody-producing cells obtained by these methods are transformed and cloned. Methods for transforming antibody-producing cells to obtain monoclonal antibodies are not limited to cell fusion. For example, methods for obtaining clonable transformants by virus infection are known.

Hybridomas that produce the monoclonal antibodies used in the present invention can be screened based on their reactivity to LY6K. Specifically, antibody-producing cells are first selected by using as an index the binding activity toward LY6K, or a domain peptide thereof, that was used as the immunogen. Positive clones that are selected by this screening are subcloned as necessary.

The monoclonal antibodies to be used in the present invention can be obtained by culturing the established hybridomas under suitable conditions and collecting the produced antibodies. When the hybridomas are homohybridomas, they can be cultured in vivo by inoculating them intraperitoneally in syngeneic animals. In this case, monoclonal antibodies are collected as ascites fluid. When heterohybridomas are used, they can be cultured in vivo using nude mice as a host.

In addition to in vivo cultures, hybridomas are also commonly cultured ex vivo, in a suitable culture environment. For example, basal media such as RPMI 1640 and DMEM are generally used as the medium for hybridomas. Additives such as animal sera can be added to these media to maintain the antibody-producing ability to a high level. When hybridomas are cultured ex vivo, the monoclonal antibodies can be collected as a culture supernatant. Culture supernatants can be collected by separating from cells after culturing, or by continuously collecting while culturing using a culture apparatus that uses a hollow fiber.

Monoclonal antibodies used in the context of the present invention can be prepared from monoclonal antibodies collected as ascites fluid or culture supernatants, by separating immunoglobulin fractions by saturated ammonium sulfate precipitation and further purifying by gel filtration, ion exchange chromatography, or such. In addition, if the monoclonal antibodies are IgGs, purification methods based on affinity chromatography with a protein A or protein G column are effective.

On the other hand, to obtain antibodies useful in the context of the present invention as polyclonal antibodies which recognize amino acid sequence comprising SEQ ID NO: 18 or 19, blood can be drawn from animals whose antibody titer increased after immunization, and the serum is separated to obtain an anti-serum. Immunoglobulins are purified from anti-sera by known methods to prepare the antibodies used in the present invention. LY6K-specific antibodies can be prepared by combining immunoaffinity chromatography which uses LY6K as a ligand with immunoglobulin purification.

When antibodies against LY6K contact LY6K, the antibodies bind to the antigenic determinant (epitope) that the antibodies recognize through an antigen-antibody reaction. Especially, the epitope comprises SEQ ID NO: 18 or 19. The binding of antibodies to antigens can be detected by various immunoassay principles. Immunoassays can be broadly categorized into heterogeneous analysis methods and homogeneous analysis methods. To maintain the sensitivity and specificity of immunoassays to a high level, monoclonal antibodies are preferred. Methods of the present invention for measuring LY6K by various immunoassay formats are described in further detail below.

First, methods for measuring LY6K using a heterogeneous immunoassay are described. In heterogeneous immunoassays, a mechanism for detecting antibodies that bound to LY6K after separating them from those that did not bind to LY6K is required.

To facilitate the separation, immobilized reagents are generally used. For example, a solid phase onto which antibodies recognizing LY6K have been immobilized is first prepared (immobilized antibodies). LY6K is made to bind to these, and secondary antibodies are further reacted thereto.

When the solid phase is separated from the liquid phase and further washed, as necessary, secondary antibodies remain on the solid phase in proportion to the concentration of LY6K. By labeling the secondary antibodies, LY6K can be quantitated by measuring the signal derived from the label.

Any method may be used to bind the antibodies to the solid phase. For example, antibodies can be physically adsorbed to hydrophobic materials such as polystyrene. Alternatively, antibodies can be chemically bound to a variety of materials having functional groups on their surfaces. Furthermore, antibodies labeled with a binding ligand can be bound to a solid phase by trapping them using a binding partner of the ligand. Combinations of a binding ligand and its binding partner include avidin-biotin and such. The solid phase and antibodies can be conjugated at the same time or before the reaction between the primary antibodies and LY6K.

Similarly, the secondary antibodies do not need to be directly labeled. That is, they can be indirectly labeled using antibodies against antibodies or using binding reactions such as that of avidin-biotin.

The concentration of LY6K in a sample is determined based on the signal intensities obtained using standard samples with known LY6K concentrations.

Any antibody can be used as the immobilized antibody and secondary antibody for the heterogeneous immunoassays mentioned above, so long as it is an antibody, or a fragment containing an antigen-binding site thereof, that recognizes LY6K. Therefore, it may be a monoclonal antibody, a polyclonal antibody, or a mixture or combination of both. For example, a combination of monoclonal antibodies and polyclonal antibodies is a preferable combination in the present invention. Alternatively, when both antibodies are monoclonal antibodies, combining monoclonal antibodies recognizing different epitopes is preferable.

In the present invention, for example, a combination of antibodies recognizing LY6K at codons 23-109 (SEQ ID NO: 18) and 71-204 (SEQ ID NO: 19) are preferable to detect LY6K with high specificity.

Since the antigens to be measured are sandwiched by antibodies, such heterogenous immunoassays are called sandwich methods. Since sandwich methods excel in the measurement sensitivity and the reproducibility, they are a preferable measurement principle in the present invention.

The principle of competitive inhibition reactions can also be applied to the heterogeneous immunoassays. Specifically, they are immunoassays based on the phenomenon where LY6K in a sample competitively inhibits the binding between LY6K with a known concentration and an antibody. The concentration of LY6K in the sample can be determined by labeling LY6K with a known concentration and measuring the amount of LY6K that reacted (or did not react) with the antibody.

A competitive reaction system is established when antigens with a known concentration and antigens in a sample are simultaneously reacted to an antibody. Furthermore, analyses by an inhibitory reaction system are possible when antibodies are reacted with antigens in a sample, and antigens with a known concentration are reacted thereafter. In both types of reaction systems, reaction systems that excel in the operability can be constructed by setting either one of the antigens with a known concentration used as a reagent component or the antibody as the labeled component, and the other one as the immobilized reagent.

Radioisotopes, fluorescent substances, luminescent substances, substances having an enzymatic activity, macroscopically observable substances, magnetically observable substances, and such are used in these heterogeneous immunoassays. Specific examples of these labeling substances are shown below.

Substances having an enzymatic activity:

peroxidase,

alkaline phosphatase,

urease, catalase,

glucose oxidase,

lactate dehydrogenase, or

amylase, etc.

Fluorescent substances:

fluorescein isothiocyanate,

tetramethylrhodamine isothiocyanate,

substituted rhodamine isothiocyanate, or

dichlorotriazine isothiocyanate, etc.

Radioisotopes:

tritium,

¹²⁵I, or

¹³¹I, etc.

Among these, non-radioactive labels such as enzymes are an advantageous label in terms of safety, operability, sensitivity, and such. Enzymatic labels can be linked to antibodies or to LY6K by known methods such as the periodic acid method or maleimide method.

As the solid phase, beads, inner walls of a container, fine particles, porous carriers, magnetic particles, or such are used. Solid phases formed using materials such as polystyrene, polycarbonate, polyvinyltoluene, polypropylene, polyethylene, polyvinyl chloride, nylon, polymethacrylate, latex, gelatin, agarose, glass, metal, ceramic, or such can be used. Solid materials in which functional groups to chemically bind antibodies and such have been introduced onto the surface of the above solid materials are also known. Known binding methods, including chemical binding such as poly-L-lysine or glutaraldehyde treatment and physical adsorption, can be applied for solid phases and antibodies (or antigens).

Although the steps of separating the solid phase from the liquid phase and the washing steps are required in all heterogeneous immunoassays exemplified herein, these steps can easily be performed using the immunochromatography method, which is a variation of the sandwich method.

Specifically, antibodies to be immobilized are immobilized onto porous carriers capable of transporting a sample solution by the capillary phenomenon, then a mixture of a sample containing LY6K and labeled antibodies is deployed therein by this capillary phenomenon. During deployment, LY6K reacts with the labeled antibodies, and when it further contacts the immobilized antibodies, it is trapped at that location. The labeled antibodies that do not react with LY6K pass through, without being trapped by the immobilized antibodies.

As a result, the presence of LY6K can be detected using, as an index, the signals of the labeled antibodies that remain at the location of the immobilized antibodies. If the labeled antibodies are maintained upstream in the porous carrier in advance, all reactions can be initiated and completed by just dripping in the sample solutions, and an extremely simple reaction system can be constructed. In the immunochromatography method, labeled components that can be distinguished macroscopically, such as colored particles, can be combined to construct an analytical device that does not even require a special reader.

Furthermore, in the immunochromatography method, the detection sensitivity for

LY6K can be adjusted. For example, by adjusting the detection sensitivity near the cutoff value described below, the aforementioned labeled components can be detected when the cutoff value is exceeded. By using such a device, whether a subject is positive or negative can be judged very simply. By adopting a constitution that allows a macroscopic distinction of the labels, necessary examination results can be obtained by simply applying blood samples to the device for immunochromatography.

Various methods for adjusting the detection sensitivity of the immunochromatography method are known. For example, a second immobilized antibody for adjusting the detection sensitivity can be placed between the position where samples are applied and the immobilized antibodies (Japanese Patent Application Kokai Publication No. (JP-A) H06-341989 (unexamined, published Japanese patent application)). LY6K in the sample is trapped by the second immobilized antibody while deploying from the position where the sample was applied to the position of the first immobilized antibody for label detection. After the second immobilized antibody is saturated, LY6K can reach the position of the first immobilized antibody located downstream. As a result, when the concentration of LY6K included in the sample exceeds a predetermined concentration, LY6K bound to the labeled antibody is detected at the position of the first immobilized antibody.

Next, homogeneous immunoassays are explained. As opposed to heterogeneous immunological assay methods that require a separation of the reaction solutions as described above, LY6K can also be measured using homogeneous analysis methods. Homogeneous analysis methods allow the detection of antigen-antibody reaction products without their separation from the reaction solutions.

A representative homogeneous analysis method is the immunoprecipitation reaction, in which antigenic substances are quantitatively analyzed by examining precipitates produced following an antigen-antibody reaction. Polyclonal antibodies are generally used for the immunoprecipitation reactions. When monoclonal antibodies are applied, multiple types of monoclonal antibodies that bind to different epitopes of LY6K are preferably used. The products of precipitation reactions that follow the immunological reactions can be macroscopically observed or can be optically measured for conversion into numerical data.

The immunological particle agglutination reaction, which uses as an index the agglutination by antigens of antibody-sensitized fine particles, is a common homogeneous analysis method. As in the aforementioned immunoprecipitation reaction, polyclonal antibodies or a combination of multiple types of monoclonal antibodies can be used in this method as well. Fine particles can be sensitized with antibodies through sensitization with a mixture of antibodies, or they can be prepared by mixing particles sensitized separately with each antibody. Fine particles obtained in this manner gives matrix-like reaction products upon contact with LY6K. The reaction products can be detected as particle aggregation. Particle aggregation may be macroscopically observed or can be optically measured for conversion into numerical data.

Immunological analysis methods based on energy transfer and enzyme channeling are known as homogeneous immunoassays. In methods utilizing energy transfer, different optical labels having a donor/acceptor relationship are linked to multiple antibodies that recognize adjacent epitopes on an antigen. When an immunological reaction takes place, the two parts approach and an energy transfer phenomenon occurs, resulting in a signal such as quenching or a change in the fluorescence wavelength. On the other hand, enzyme channeling utilizes labels for multiple antibodies that bind to adjacent epitopes, in which the labels are a combination of enzymes having a relationship such that the reaction product of one enzyme is the substrate of another. When the two parts approach due to an immunological reaction, the enzyme reactions are promoted; therefore, their binding can be detected as a change in the enzyme reaction rate.

In the present invention, blood for measuring LY6K can be prepared from blood drawn from patients. Preferable blood samples are the serum or plasma. Serum or plasma samples can be diluted before the measurements. Alternatively, the whole blood can be measured as a sample and the obtained measured value can be corrected to determine the serum concentration. For example, concentration in whole blood can be corrected to the serum concentration by determining the percentage of corpuscular volume in the same blood sample.

In a preferred embodiment, the immunoassay is an ELISA. The present invention further provides sandwich ELISA to detect serum LY6K in patients with lung cancer or esophageal cancer.

The LY6K level in the blood samples is then compared with an LY6K level associated with a reference sample such as a normal control sample. The phrase “normal control level” refers to the level of LY6K typically found in a blood sample of a population not suffering from lung cancer or esophageal cancer. The reference sample is preferably of a similar nature to that of the test sample. For example, if the test sample is composed of patient serum, the reference sample should also be serum. The LY6K level in the blood samples from control and test subjects may be determined at the same time or, alternatively, the normal control level may be determined by a statistical method based on the results obtained by analyzing the level of LY6K in samples previously collected from a control group.

The LY6K level may also be used to monitor the course of treatment of lung cancer or esophageal cancer. In this method, a test blood sample is provided from a subject undergoing treatment for lung cancer or esophageal cancer. Preferably, multiple test blood samples are obtained from the subject at various time points before, during, or after the treatment. The level of LY6K in the post-treatment sample may then be compared with the level of LY6K in the pre-treatment sample or, alternatively, with a reference sample (e.g., a normal control level). For example, if the post-treatment LY6K level is lower than the pre-treatment LY6K level, one can conclude that the treatment was efficacious. Likewise, if the post-treatment LY6K level is similar to the normal control LY6K level, one can also conclude that the treatment was efficacious.

An “efficacious” treatment is one that leads to a reduction in the level of LY6K or a decrease in size, prevalence, or metastatic potential of lung cancer or esophageal cancer in a subject. When a treatment is applied prophylactically, “efficacious” means that the treatment retards or prevents occurrence of lung cancer or esophageal cancer or alleviates a clinical symptom of lung cancer or esophageal cancer. The assessment of lung cancer or esophageal cancer can be made using standard clinical protocols. Furthermore, the efficaciousness of a treatment can be determined in association with any known method for diagnosing or treating lung cancer or esophageal cancer. For example, lung cancer or esophageal cancer is routinely diagnosed histopathologically or by identifying symptomatic anomalies.

Therefore, the possibility that a patient judged to have lung cancer or esophageal cancer based on LY6K or a combination of LY6K with CEA and/or CYFRA 21-1 can be easily ruled out.

Components used to carry out the diagnosis of cancers such as lung cancer and esophageal cancer according to the present invention can be combined in advance and supplied as a testing kit. Accordingly, the present invention provides a kit for detecting lung cancer or esophageal cancer, including:

(i) an immunoassay reagent for determining a level of LY6K in a blood sample; and

(ii) a positive control sample for LY6K.

In the preferable embodiments, the kit of the present invention may further include:

(iii) an immunoassay reagent for determining either of the levels of CEA and CYFRA 21-1 or both in a blood sample; and

(iv) a positive control sample for CEA or CYFRA 21-1.

The reagents for the immunoassays which constitute a kit of the present invention may include reagents necessary for the various immunoassays described above. Specifically, the reagents for the immunoassays include an antibody that recognizes the substance to be measured. Especially, the antibody recognizes amino acid sequence comprising SEQ ID NO: 18 or 19. The antibody can be modified depending on the assay format of the immunoassay. ELISA can be used as a preferable assay format of the present invention. In ELISA, for example, a first antibody immobilized onto a solid phase and a second antibody having a label are generally used.

Therefore, the immunoassay reagents for ELISA can include a first antibody immobilized onto a solid phase carrier. Fine particles or the inner walls of a reaction container can be used as the solid phase carrier. Magnetic particles can be used as the fine particles. Alternatively, multi-well plates such as 96-well microplates are often used as the reaction containers. Containers for processing a large number of samples, which are equipped with wells having a smaller volume than in 96-well microplates at a high density, are also known. In the present invention, the inner walls of these reaction containers can be used as the solid phase carriers.

The immunoassay reagents for ELISA may further include a second antibody having a label. The second antibody for ELISA may be an antibody onto which an enzyme is directly or indirectly linked. Methods for chemically linking an enzyme to an antibody are known. For example, immmunoglobulins can be enzymatically cleaved to obtain fragments that include the variable regions. By reducing the —SS— bonds contained in these fragments to —SH groups, bifunctional linkers can be attached. By linking an enzyme to the bifunctional linkers in advance, enzymes can be linked to the antibody fragments.

Alternatively, to indirectly link an enzyme, for example, the avidin-biotin binding can be used. That is, an enzyme can be indirectly linked to an antibody by contacting a biotinylated antibody with an enzyme to which avidin has been attached. In addition, an enzyme can be indirectly linked to a second antibody using a third antibody which is an enzyme-labeled antibody recognizing the second antibody. For example, enzymes such as those exemplified above can be used as the enzymes to label the antibodies.

Kits of the present invention may include a positive control for LY6K. A positive control for LY6K includes LY6K whose concentration has been determined in advance. For example, a control sample whose LY6K concentration is higher than the cut-off value may be used as the positive control. Alternatively, preferable concentrations are, for example, a concentration set as the standard value in a testing method of the present invention. Further, a positive control having a higher concentration can also be combined. The positive control for LY6K in the present invention can additionally include CEA or CYFRA 21-1 whose concentration has been determined in advance. A positive control including LY6K and CEA and/or CYFRA 21-1 is preferable as the positive control of the present invention.

Therefore, the present invention provides a positive control for detecting cancers such as lung cancer and esophageal cancer which includes LY6K and CEA and/or CYFRA 21-1 at concentrations above a normal value. Alternatively, the present invention relates to the use of a blood sample comprising LY6K and CEA and/or CYFRA 21-1 at concentrations above a normal value in the production of a positive control for the detection of lung cancer or esophageal cancer. It has been known that CEA or CYFRA 21-1 can serve as an index for lung cancer or esophageal cancer; however, that LY6K can serve as an index for lung cancer or esophageal cancer is a novel finding obtained by the present invention. Therefore, positive controls including LY6K and CEA and/or CYFRA 21-1 are novel. The positive controls of the present invention can be prepared by adding LY6K and CEA and/or CYFRA 21-1 at concentrations above a standard value to blood samples. For example, sera including LY6K and CEA and/or CYFRA 21-1 at concentrations above a standard value are preferable as the positive controls of the present invention.

The positive controls in the present invention are preferably in a liquid form. In the present invention, blood samples are used as samples. Therefore, samples used as controls also need to be in a liquid form. Alternatively, by dissolving a dried positive control with a predefined amount of liquid at the time of use, a control that gives the tested concentration can be prepared. By packaging, together with a dried positive control, an amount of liquid necessary to dissolve it, the user can obtain the necessary positive control by just mixing them. LY6K used as the positive control can be a naturally-derived protein or it may be a recombinant protein. Not only positive controls, but also negative controls can be combined in the kits of the present invention. The positive controls or negative controls are used to verify that the results indicated by the immunoassays are correct.

Method for Assessing the Prognosis of Cancer

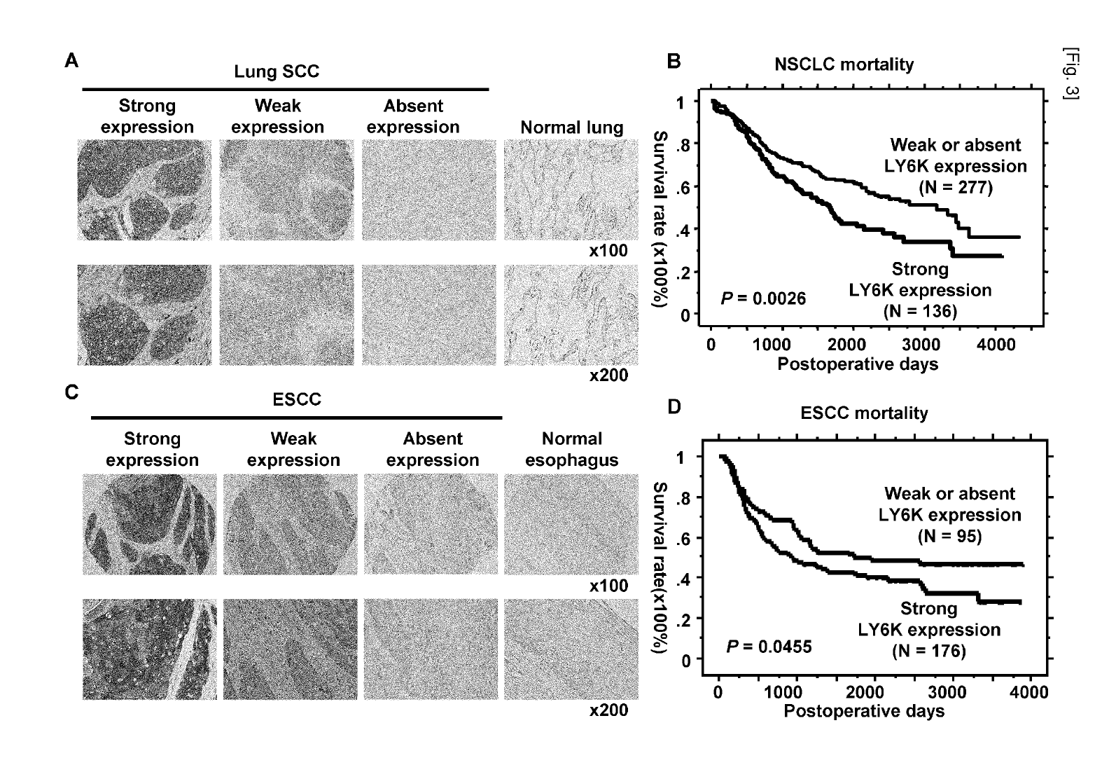

According to the present invention, it was newly discovered that LY6K expression is significantly associated with poorer prognosis in patients (see FIG. 3B and D). Thus, the present invention provides a method for determining or assessing the prognosis of a patient with cancer, in particular, esophageal and/or lung cancer, by detecting the expression level of the LY6K gene in a biological sample of the patient; comparing the detected expression level to a control level; and correlating an increased expression level to the control level with an indication of poor prognosis (poor survival). Alternatively, according to the present invention, an intermediate result for determining or assessing the prognosis of a subject may be provided. Such intermediate result may be combined with additional information to assist a doctor, nurse, or other practitioner to determine or assess the prognosis of a patient with cancer. Alternatively, the present invention may be used to detect cancerous cells in a subject-derived tissue, and provide a doctor with useful information to determine or assess the prognosis of a patient with cancer.

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of LY6K over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the LY6K gene can be detected in the sample. Preferably, the biological sample is an esophageal and lung cell (a cell obtained from the esophagus and lung). Furthermore, the biological sample includes bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the LY6K gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the LY6K gene detected before any kind of treatment in an individual or a population of individuals who showed good or positive prognosis of cancer, after the treatment, which herein will be referred to as “good prognosis control level”. Alternatively, the “control level” may be the expression level of the LY6K gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level may be determined based on the expression level of the LY6K gene detected before any kind of treatment in a patient of cancer, or a population of the patients whose disease state (good or poor prognosis) is known. Preferably, cancer is esophageal or lung cancer. It is preferable to use the standard value of the expression levels of the LY6K gene in a patient group with a known disease state. The standard value may be obtained by any method known in the art. For example, a range of mean+/−2S.D. or mean+/−3S.D. may be used as standard value.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the LY6K gene in samples previously collected and stored from a control group. Furthermore, the control level can be a database of expression patterns obtained from previously tested cells. Moreover, according to an aspect of the present invention, the expression level of the LY6K gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the LY6K gene relative to the good prognosis control level indicates a more favorable prognosis of the patient and an increase in the expression level relative to the good prognosis control level indicates less favorable, poorer prognosis for post-treatment remission, recovery, survival, and/or clinical outcome. On the other hand, a decrease in the expression level of the LY6K gene relative to the poor prognosis control level indicates a more favorable prognosis of the patient and a similarity in the expression level relative to the poor prognosis control level indicates less favorable, poorer prognosis for post-treatment remission, recovery, survival, and/or clinical outcome.

An expression level of the LY6K gene in a biological sample can be considered altered when the expression level differs from the control level by more than 1.0, 1.5, 2.0, 5.0, 10.0, or more fold. Alternatively, an expression level of the LY6K gene in a biological sample can be considered altered, when the expression level is increased or decreased to the control level at least 10%, 20%, 30%, 40%, 50%, 60%, 80%, 90%, or more.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the LY6K gene.

The expression level may be determined by detecting the gene transcript in the patient-derived biological sample using techniques well known in the art. The gene transcripts detected by the present method include both the transcription and translation products, such as mRNA and protein.

For instance, the transcription product of the LY6K gene can be detected by hybridization, e.g., Northern blot hybridization analyses, that use an LY6K gene probe to the gene transcript. The detection may be carried out on a chip or an array. The use of an array is preferable for detecting the expression level of a plurality of genes including the LY6K gene. As another example, amplification-based detection methods, such as reverse-transcription based polymerase chain reaction (RT-PCR) which use primers specific to the LY6K gene may be employed for the detection (see Example). The LY6K gene-specific probe or primers may be designed and prepared using conventional techniques by referring to the whole sequence of the LY6K gene (SEQ ID NO: 1). For example, the primer sets (SEQ ID NOs: 3 and 4, 7 and 4) used in the Example may be employed for the detection by RT-PCR, but the present invention is not restricted thereto.

Specifically, a probe or primer used for the present method hybridizes under stringent, moderately stringent, or low stringent conditions to the mRNA of the LY6K gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the assessment of the present invention. For example, the quantity of the LY6K protein may be determined. A method for determining the quantity of the protein as the translation product includes immunoassay methods that use an antibody specifically recognizing the LY6K protein. The antibody may be monoclonal or polyclonal. Especially, the antibody recognizes amino acid sequence comprising SEQ ID NO: 18 or 19. Furthermore, any fragment or modification (e.g., chimeric antibody, scFv, Fab, F(ab′)2, Fv, etc.) of the antibody may be used for the detection, so long as the fragment retains the binding ability to the LY6K protein. Methods to prepare these kinds of antibodies for the detection of proteins are well known in the art, and any method may be employed in the present invention to prepare such antibodies and equivalents thereof.

As another method to detect the expression level of the LY6K gene based on its translation product, the intensity of staining may be observed via immunohistochemical analysis using an antibody against LY6K protein. Namely, the observation of strong staining indicates an increased presence of the LY6K protein and at the same time high expression level of the LY6K gene.

Furthermore, the LY6K protein is known to have a cell proliferating activity.

Therefore, the expression level of the LY6K gene can be determined using such cell proliferating activity as an index. For example, cells from a biological sample are prepared and cultured, and then by detecting the speed of proliferation, or by measuring the cell cycle or the colony forming ability, the expression level of the LY6K gene can be determined.

Moreover, in addition to the expression level of the LY6K gene, the expression level of other esophageal and lung cell-associated genes, for example, genes known to be differentially expressed in esophageal and lung cancer, may also be determined to improve the accuracy of the assessment. Examples of other lung cell-associated genes include, but are not limited to, those described in the WIPO Publication WO 2004/031413, the entire contents of which are incorporated by reference herein. In this publication, LY6K is referred to as URLC10.

The patient to be assessed for the prognosis of cancer according to the method is preferably a mammal and includes human, non-human primate, mouse, rat, dog, cat, horse, and cow.

A Kit for Diagnosing Cancer and Assessing the Prognosis of Cancer

Kits:

The present invention provides a kit for diagnosing cancer or assessing the prognosis of cancer, preferably, esophageal or lung cancer. Specifically, the kit contains at least one reagent for detecting the expression of the LY6K gene in a patient-derived biological sample, which reagent may be selected from the group of:

(a) a reagent for detecting mRNA of the LY6K gene;

(b) a reagent for detecting the LY6K protein; and

(c) a reagent for detecting the biological activity of the LY6K protein.

Suitable reagents for detecting mRNA of the LY6K gene include nucleic acids that specifically bind to or identify the LY6K mRNA, such as oligonucleotides which have a complementary sequence to a part of the LY6K mRNA. These kinds of oligonucleotides are exemplified by primers and probes that are specific to the LY6K mRNA. These kinds of oligonucleotides may be prepared based on methods well known in the art. If needed, the reagent for detecting the LY6K mRNA may be immobilized on a solid matrix. Moreover, more than one reagent for detecting the LY6K mRNA may be included in the kit.

On the other hand, suitable reagents for detecting the LY6K protein include antibodies to the LY6K protein. The antibody may be monoclonal or polyclonal, e.g., TM38 and MB44 which recognize LY6K at codons 23-109 (SEQ ID NO: 18) and 71-204 (SEQ ID NO: 19), respectively. These two highly LY6K-specific antibodies recognize different epitopes of the LY6K protein, and can be suitably used as the primary and secondary antibodies in sandwich ELISA assays in the present invention. Furthermore, any fragmental or modified version (e.g., chimeric antibody, scFv, Fab, F(ab′)2, Fv, etc.) of the antibody may be used as the reagent, so long as the fragment retains the binding ability to the LY6K protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the LY6K protein may be included in the kit.

Furthermore, when a cell expressing LY6K, the biological activity can be determined by, for example, measuring the cell proliferating activity due to the expressed LY6K protein. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the LY6K mRNA may be immobilized on a solid matrix. Moreover, more than one reagent for detecting the biological activity of the LY6K protein may be included in the kit.

The kit may contain more than one of the aforementioned reagents. Furthermore, the kit may contain a solid matrix and reagent for binding a probe against the LY6K gene or antibody against the LY6K protein, a medium and container for culturing cells, positive and negative control reagents, and a secondary antibody for detecting an antibody against the LY6K protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be included in a container with a label. Suitable containers include bottles, vials, and test tubes. The containers may be formed from a variety of materials, such as glass or plastic.

The assay format of the kit can be a Northern hybridization or a sandwich ELISA, both of which are known in the art. See, for example, Sambrook and Russell, Molecular Cloning: A Laboratory Manual, 3^{rd} Edition, 2001, Cold Spring Harbor Laboratory Press; and Using Antibodies, supra.

For example, an LY6K detection reagent can be immobilized on a solid matrix, for example a porous strip, to form at least one LY6K detection site. The measurement or detection region of the porous strip can include a plurality of sites, each containing a nucleic acid. A test strip can also contain sites for negative and/or positive controls. Alternatively, control sites can be located on a separate strip from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of LY6K present in the sample. The detection sites can be configured in any suitably detectable shape and are typically in the shape of a bar or dot spanning the width of a test strip.

As an embodiment of the present invention, when the reagent is a probe against the LY6K mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of LY6K mRNA present in the sample. The detection sites may be configured in any suitably detectable shape and are typically in the shape of a bar or dot spanning the width of a test strip.

The kit of the present invention may further contain a positive control sample or LY6K standard sample. The positive control sample of the present invention may be prepared by collecting LY6K positive blood samples and then those LY6K level are assayed. Alternatively, purified LY6K protein or polynucleotide may be added to LY6K free serum to form the positive sample or the LY6K standard. In the present invention, purified LY6K may be recombinant protein. The LY6K level of the positive control sample is, for example more than cut off value.

Furthermore, the present invention provides a kit containing at least one reagent for detecting the expression of the LY6K gene and one or more reagent for detecting the expression of other cancer-associated proteins in a patient-derived biological sample. Suitable reagents for detecting the other cancer-associated proteins include antibodies to the other cancer-associated proteins, e.g., ELISA. For example, the levels of CEA in serum were measured by ELISA with a commercially available enzyme test kit (HOPE Laboratories, Belmont, Calif.), according to the supplier's recommendations, the levels of CYFRA 21-1 in serum were measured by ELISA with a commercially available kit (DRG, Marburg, Germany).

Hereinafter, the present invention is described in more detail by reference to the

Examples. However, the following materials, methods and examples only illustrate aspects of the invention and in no way are intended to limit the scope of the present invention. As such, methods and materials similar or equivalent to those described herein can be used in the practice or testing of the present invention.

### Examples

### Example 1

Materials and Methods

Cell Lines:

The 5 human NSCLC cell lines used in this study included three adenocarcinoma cell lines (ADCs; A427, LC319 and NCI-H1373), two squamous-cell carcinoma cell lines (SCCs; RERF-LC-AI and NCI-H226) (Hammarstrom S. Semin Cancer Biol. 1999 April;9(2):67-81.). All cells were grown in monolayers in appropriate media supplemented with 10% fetal calf serum (FCS) and were maintained at 37 degrees C. in an atmosphere of humidified air with 5% CO₂. Human small airway epithelial cells (SAEC) were grown in optimized medium (SAGM) purchased from Cambrex Bio Science Inc. Primary NSCLC and ESCC samples had been obtained earlier with informed consent (Taniwaki M, et al, Int J Oncol. 2006 September;29(3):567-75; Yamabuki T, et al, Int J Oncol. 2006 June;28(6):1375-84; Ishikawa N, et al. Cancer Sci. 2006 August;97(8):737-45.).

A total of 413 formalin-fixed samples of primary NSCLCs (259 ADCs, 113 SCCs, 28 LCCs, 13 ASCs; 129 female and 284 male patients; median age of 64.5 with a range of 26-84 years), and adjacent normal lung tissues, had been obtained earlier along with clinicopathological data from patients undergoing curative surgery.

A total of 271 formalin-fixed primary ESCCs (26 female and 245 male patients; median age of 61.4+/−8.1 SD with a range of 38-77 years) and adjacent normal esophageal tissue samples had also been obtained from patients undergoing curative surgery. NSCLC specimen and five tissues (heart, liver, lung, kidney, and testis) from post-mortem materials (2 individuals with SCC) were also obtained.

The pathological stage was determined according to the classification of the Union

Internationale Controle Cancer (Travis W D, et al., World Health Organization International Histological classification of tumours 1999.). This study and the use of all clinical materials mentioned were approved by individual institutional Ethical Committees.

Serum Samples:

Serum samples were obtained with informed consent from 74 healthy individuals as controls (14 females and 60 males; median age 48.0+/−7.47 SD with a range of 33-60 years), and from 65 non-neoplastic lung disease patients with chronic obstructive pulmonary disease (COPD) enrolled as a part of the Japanese Project for Personalized Medicine (BioBank Japan) or admitted to Hiroshima University Hospital (8 females and 57 males; median age of 66.0+/−5.92 SD with a range of 54-73 years). All of these patients were current and/or former smokers (The mean[+/−1SD] of pack-year index (PYI) was 55.6+/−50.1 SD; PYI was defined as the number of cigarette packs [20 cigarette per pack] consumed a day multiplied by years).

Serum samples were also obtained from 112 NSCLC patients (40 females and 72 males; median age 66.0+/−12.0 SD with a range of 30-84) and 81 esophageal-cancer patients (12 females and 69 males; median age 65.0+/−5.1 SD with a range of 37-74). These 112 NSCLC cases included 85 ADCs and 27 SCCs. Samples were selected for the study on the basis of the following criteria:

(1) patients were newly diagnosed and previously untreated and

(2) their tumors were pathologically diagnosed as lung or esophageal cancers (stages I-IV). Serum was obtained at the time of diagnosis and stored at −150 degrees C.

Semi-Quantitative RT-PCR:

Total RNA was extracted from cultured cells and clinical tissues using Trizol reagent (Life Technologies, Inc. Gaithersburg, Md.) according to the manufacturer's protocol. Extracted RNAs and normal human-tissue poly(A) RNAs were treated with DNase I (Roche Diagnostics, Basel, Switzerland) and then reverse-transcribed using oligo (dT)12-18 primer and SuperScript II reverse transcriptase (Life Technologies, Inc.). The nucleotide sequences of the primers for the semi-quantitative RT-PCR experiments are follows:

| LY6K gene-specific primers | |
|---|---|
| 5′-ATTCGCTACTGCAATTTAGAGG-3′ | (SEQ ID NO: 3) |
| and | |
| 5′-GTTTAATGCAACAGGTGACAACG-3′, | (SEQ ID NO: 4)) |
| beta-actin (ACTB)-specific primers | |
| 5′-GAGGTGATAGCATTGCTTTCG-3′ | (SEQ ID NO: 5) |
| and | |
| 5′-CAAGTCAGTGTACAGGTAAGC-3′. | (SEQ ID NO: 6)) |

All PCR reactions involved initial denaturation at 94 degrees C. for 2 min followed by 22 (for ACTB) or 30 cycles (for LY6K) of 94 degrees C. 30 s, 58 degrees C. for 30 s, and 72 degrees C. for 60 s on a GeneAmp PCR system 9700 (Applied Biosystems, Foster City, Calif.).

Northern-Blot Analysis:

Human multiple-tissue blots (BD Biosciences, Palo Alto, Calif.) were hybridized with ₃₂P-labeled PCR products. PCR product of LY6K was prepared as a probe by RT-PCR using primers 5′-AGGGTGACAATAGAGTGTGGTGT-3′ (SEQ ID NO: 7) and 5′-GTTTAATGCAACAGGTGACAACG-3′ (SEQ ID NO: 4). Prehybridization, hybridization, and washing were performed according to the supplier's recommendations. The blots were autoradiographed with intensifying screens at −80 degrees C. for one week.

RNA Interference Assay:

A vector-based RNA interference (RNAi) system, psiH1BX3.0, had been previously established to direct the synthesis of siRNAs in mammalian cells (Suzuki C, et al. Cancer Res. 2003 Nov. 1;63(21):7038-41; Kato T, et al. Cancer Res. 2005 Jul. 1;65(13):5638-46.). 10 mcg of siRNA-expression vector, using 30 mcl of Lipofectamine 2000 (Invitrogen, Carlsbad, Calif.), was transfected into lung-cancer cell lines, which over-expressed LY6K. The transfected cells were cultured for five days in the presence of appropriate concentrations of geneticin (G418), and then cell numbers and viability were measured by Giemsa staining and triplicate MTT assays. The target sequences of the synthetic oligonucleotides for RNAi were as follows: control 1 (EGFP: enhanced green fluorescent protein (GFP) gene, a mutant of Aequorea victoria GFP), 5′-GAAGCAGCACGACTTCTTC-3′ (SEQ ID NO: 8); control 2 (Scramble (SCR): chloroplast Euglena gracilis gene coding for 5S and 16S rRNAs), 5′-GCGCGCTTTGTAGGATTCG-3′ (SEQ ID NO: 9);

LY6K siRNA-1 (si-LY6K-1), 5′-AACCTGACTGCGAGACAACGA-3′ (SEQ ID NO: 10) (at the position of 473-493 nt of SEQ ID NO: 1);

LY6K siRNA-2 (si-LY6K-2), 5′-AAGGAGGTGCAAATGGACAGA-3′ (SEQ ID NO: 11) (at the position if 586-606 nt of SEQ ID NO: 1). Down-regulation of LY6K protein expression by effective siRNA (si-LY6K-2), but not by the two controls or si-LY6K-1, was confirmed with western-blotting in the cell lines used for this assay.

| | | SEQ | |
|---|---|---|---|
| | | ID | |
| clone | | NO | sequence |
| si-LY6K-1 | target | 10 | AACCTGACTGCGAGACAACGA |
| | oligo | 12 | TCCCAACCTGACTGCGAGACAACGATTC |
| | sense | | AAGAGATCGTTGTCTCGCAGTCAGGTT |
| | oligo | 13 | AAAAAACCTGACTGCGAGACAACGATCT |
| | antisense | | CTTGAATCGTTGTCTCGCAGTCAGGTT |
| | hairpin | 14 | AACCTGACTGCGAGACAACGATTCAAGA |
| | | | GATCGTTGTCTCGCAGTCAGGTT |
| si-LY6K-2 | target | 11 | AAGGAGGTGCAAATGGACAGA |
| | oligo | 15 | TCCCAAGGAGGTGCAAATGGACAGATTC |
| | sense | | AAGAGATCTGTCCATTTGCACCTCCTT |
| | oligo | 16 | AAAAAAGGAGGTGCAAATGGACAGATCT |
| | antisense | | CTTGAATCTGTCCATTTGCACCTCCTT |
| | hairpin | 17 | AAGGAGGTGCAAATGGACAGATTCAAGA |
| | | | GATCTGTCCATTTGCACCTCCTT |

Preparation of Anti-LY6K Polyclonal Antibody:

Two types of rabbit antibodies termed TM38 and MB44 specific for LY6K were raised by immunizing rabbits with 6-histidine fused human LY6K protein (codons 23-109 (SEQ ID NO: 18) and 71-204 (SEQ ID NO: 19), respectively), and purified with standard protocols using affinity columns (Affi-gel 10; Bio-Rad Laboratories, Hercules, Calif.) conjugated with the 6-histidine fused protein. On western blots, it was confirmed that the antibodies were specific for LY6K, using lysates from NSCLC tissues and cell lines as well as normal lung tissues.

Western-Blotting:

An ECL western-blotting analysis system (GE Healthcare Bio-sciences, Piscataway, N.J.) was used. SDS-PAGE was performed in 7.5% polyacrylamide gels. PAGE-separated proteins were electro-blotted onto nitrocellulose membranes (GE Healthcare Bio-sciences) and incubated with a rabbit polyclonal anti-human LY6K antibody. A goat anti-rabbit IgG-HRP antibody (GE Healthcare Bio-sciences) was served as the secondary antibodies for these experiments.

Immunohistochemistry and Tissue Microarray:

Tumor-tissue microarrays were constructed using 413 formalin-fixed primary NSCLCs and 271 ESCCs, as published previously (Chin S F, et al. Mol Pathol. 2003 October;56(5):275-9; Callagy G, et al. Diagn Mol Pathol. 2003 March;12(1):27-34; Callagy G, et al. J Pathol. 2005 February;205(3):388-96.). Briefly, the tissue area for sampling was selected based on visual alignment with the corresponding HE-stained section on a slide. Three, four, or five tissue cores (diameter 0.6 mm; height 3-4 mm) taken from a donor tumor block were placed into a recipient paraffin block using a tissue microarrayer (Beecher Instruments, Sun Prairie, Wis.). A core of normal tissue was punched from each case, and 5-micrometer sections of the resulting microarray block were used for immunohistochemical analysis.

To investigate the status of the LY6K protein in clinical lung-cancer samples that had been embedded in paraffin blocks, the sections were stained in the following manner. Briefly, a rabbit polyclonal anti-human LY6K antibody (TM38) was added after blocking of endogenous peroxidase and proteins. The sections were incubated with HRP-labeled anti-rabbit IgG as the secondary antibody. Substrate-chromogen was added and the specimens were counterstained with hematoxylin.

Three independent investigators assessed LY6K positivity semi-quantitatively without prior knowledge of clinicopathological data. The intensity of LY6K staining was evaluated using following criteria: strong positive (2+), dark brown staining in more than 50% of tumor cells completely obscuring membrane and cytoplasm; weak positive (1+), any lesser degree of brown staining appreciable in tumor cell membrane and cytoplasm; absent (scored as 0), no appreciable staining in tumor cells. Cases were accepted only as strongly positive if reviewers independently defined them as such.

Statistical Analysis:

Contingency tables were used to analyze the relationship of LY6K expression levels and clinicopathological variables of NSCLC or ESCC patients. Tumor-specific survival curves were calculated from the date of surgery to the time of death related to NSCLC or ESCC, or to the last follow-up observation. Kaplan-Meier curves were calculated for each relevant variable and for LY6K expression; differences in survival times among patient subgroups were analyzed using the log-rank test.

Univariate and multivariate analyses were performed with the Cox proportional-hazard regression model to determine associations between clinicopathological variables and cancer-related mortality. First, associations were analyzed between death and possible prognostic factors including age, gender, histological type, pT-classification, and pN-classification, taking into consideration one factor at a time. Second, multivariate Cox analysis was applied on backward (stepwise) procedures that always forced LY6K expression into the model, along with any and all variables that satisfied an entry level of a p value smaller than 0.05. As the model continued to add factors, independent factors did not exceed an exit level of P<0.05.

ELISA:

Serum levels of LY6K were measured by sandwich-type ELISA which had been originally constructed. In brief, for detection of soluble LY6K in serum, 96-well flexible microtiter plates (439454; NALGE NUNC International, Rochester, N.Y.) were coated with 2 ng/ml of capturing polyclonal antibody to LY6K (TM38) overnight. Wells were blocked with 200 mcl PBS (pH 7.4) containing 1% BSA, 5% sucrose, and 0.05% NaN3 for 2 hours and then incubated for 2 hours with 3-fold diluted serum samples in PBS (pH 7.4) containing 1% BSA. After washing with PBS (pH 7.4) containing 0.05% Tween 20, the wells were incubated for 2 hours with 200 ng/ml of biotin-conjugated polyclonal anti-LY6K antibody (MB44), followed by reaction with avidin-conjugated peroxidase (P347; Dako Cytomation, Glostrup, Denmark) for 30 minutes using a Substrate Reagent (R&D Systems).

To prepare biotinylating rabbit polyclonal antibodies to LY6K (MB44), the Biotin Labeling Kit-NH2 (LK03) was used according to the supplier's protocol (DOJINDO LABORATORIES, Kumamoto, Japan). The color reaction was stopped by adding 100 mcl of 2N sulfuric acid. Color intensity was determined by a photometer at a wavelength of 450 nm, with a reference wave-length of 570 nm. A standard curve was drawn for each plate using recombinant LY6K proteins as a reference. Levels of CEA in serum were measured by ELISA with a commercially available enzyme test kit (HOPE Laboratories, Belmont, Calif.), according to the supplier's recommendations. Levels of CYFRA 21-1 in serum were measured by ELISA with a commercially available kit (DRG, Marburg, Germany).

Differences in the levels of LY6K, CEA, and CYFRA 21-1 between tumor groups and a healthy control group were analyzed by Mann-Whitney U tests. The levels of LY6K, CEA, and CYFRA 21-1 were further evaluated by receiver-operating characteristic (ROC) curve analysis to determine cut-off levels with optimal diagnostic accuracy and likelihood ratios. The correlation coefficients between LY6K and CEA/CYFRA 21-1 were calculated with Spearman rank correlation. Significance was defined as P<0.05.

### Example 2

LY6K Expression in Lung and Esophageal Tumors, Cell Lines, and Normal Tissues.

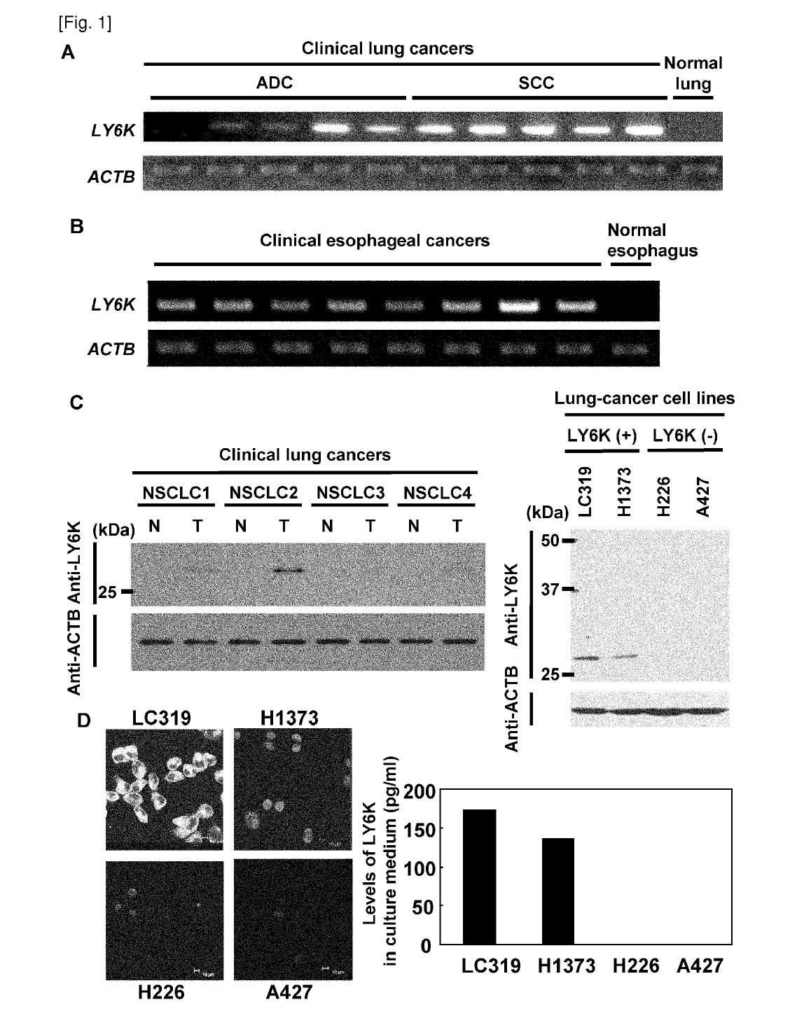

To search for novel molecules to serve as diagnostic biomarkers and/or targets for development of therapeutic agents for lung and esophageal cancers, cDNA microarray analyses were applied to search for candidate genes that were transactivated in a large proportion of NSCLCs. Among 27,648 genes screened, the LY6K transcript was identified as expressed specifically in the great majority of the lung and esophageal cancer samples examined. Its transactivation was confirmed by semi-quantitative RT-PCR experiments in 9 of 10 additional NSCLC tissues, in 8 of 8 ESCC tissues (FIGS. 1A and 1B).

Rabbit polyclonal antibody specific for human LY6K was subsequently generated and used to confirm by western-blot analysis an expression of LY6K protein in NSCLC samples in four representative pairs of NSCLC tissues and in four lung-cancer cell lines (two LY6K-positive and two LY6K-negative cell lines) (FIG. 1C). The Immunofluorescence analysis was performed to examine the subcellular localization of endogenous LY6K in the four lung-cancer cell lines (LC319, NCI-H1373, NCI-H226, and A427), and found that LY6K was located at cytoplasm of tumor cells with granular appearance (FIG. 1D, left panels).

Since LY6K encodes GPI-anchored cell surface protein and some of GPI-anchored proteins were known to be secreted into extra cellular space (Nakatsura T, et al. Biochem Biophys Res Commun. 2003 Jun. 20;306(1):16-25.), its presence in the culture media of the lung-cancer cell lines was examined by ELISA. The amounts of detectable LY6K in the culture media was concordant to the expression levels of LY6K detected with semi-quantitative RT-PCR and western-blot analyses (FIG. 1D, right panel).

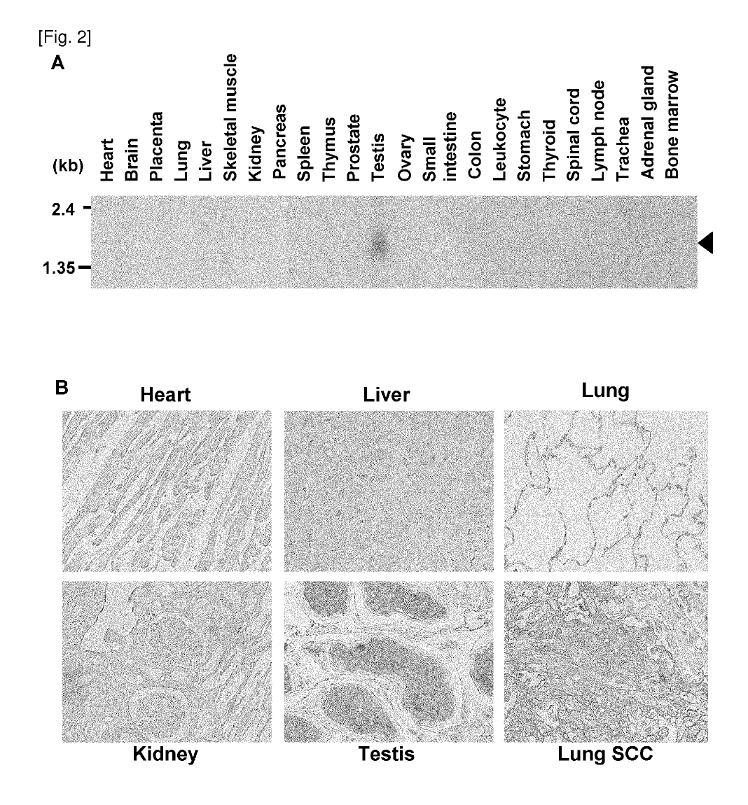

Northern blot analysis using an LY6K cDNA fragment as the probe identified a transcript of about 1.8-kb that was highly and exclusively expressed in testis among 23 normal human tissues examined (FIG. 2A). Expression of LY6K protein was subsequently examined in five normal tissues (heart, liver, lung, kidney, and testis) as well as lung cancers using anti-LY6K antibody, and found that it was hardly detectable in the former four tissues while positive LY6K staining appeared in testis and lung tumor tissues (FIG. 2B).

### Example 3

Association of LY6K Over-Expression with Poor Clinical Outcomes among NSCLC and ESCC Patients.

To verify the biological and clinicopathological significance of LY6K, the expression of LY6K protein was examined by means of tissue microarrays consisting of 413 NSCLC and 271 ESCC cases who underwent curative surgical resection. LY6K staining was observed mainly in the cell membrane and cytoplasm of tumor cells, but was hardly detectable in surrounding normal tissues (FIGS. 3A and C).

A pattern of LY6K expression was classified on the tissue array ranging from absent/weak (scored as 0-1+) to strong (2+). Positive staining was observed in 224 (86.5%) of 259 lung ADC cases, 104 (92.0%) of 113 lung SCCs, 24 (85.7%) of 28 lung LCCs, and 13 (100%) of 13 lung ASCs, while no staining was observed in any of the normal portions of the same tissues. Of the 413 NSCLC cases examined, LY6K was strongly stained in 136 (32.9%; score 2+), weakly stained in 229 (55.5%; score 1+), and not stained in 48 cases (11.6%; score 0) (details are shown in Table 2A). NSCLC patients whose tumors showed strong LY6K expression revealed shorter tumor-specific survival compared to those with absent/weak LY6K expression (P=0.0026 by log-rank test; FIG. 3B).

Univariate analysis was also applied to evaluate associations between patient prognosis and other factors including age (<65 versus 65>=), gender (female versus male), histological type (ADC versus non-ADC), pT classification (T1, T2 vs T3, 4), pN classification (N0 versus N1, N2), and LY6K status (0, 1+versus 2+). Among those parameters, LY6K status (P=0.0028), elderly (P=0.0081), male (P=0.0022), non-ADC histological classification (P=0.0090), advanced pT stage (P<0.0001), and advanced pN stage (P<0.0001) were significantly associated with poor prognosis (Table 2B). In multivariate analysis of the prognostic factors, strong LY6K expression, elderly, male gender, higher pT stage, and higher pN stage were indicated to be an independent prognostic factor (P=0.0201, <0.0001, 0.0166, 0.0002, and <0.0001, respectively; Table 2B).

Positive staining was observed in 257 (94.8%) of 271 esophageal cancer, while no staining was observed in any of the normal portions of the same tissues. LY6K was strongly stained in 176 (64.9%; score 2+), weakly stained in 81 (29.9%; score 1+), and not stained in 14 cases (5.2%; score 0) (details are shown in Table 3A). The median survival time of ESCC patients was significantly shorter in accordance with the higher expression levels of LY6K (P=0.0455 by log-rank test; FIG. 3D).

Univariate analysis was also applied to evaluate associations between ESCC patient prognosis and several factors including age (<65 versus 65>=), gender (female versus male), pT stage (tumor depth; T1+T2 versus T3+T4), pN stage (node status; N0 versus N1), and LY6K status (score 0, 1+versus 2+). Among those parameters, LY6K status (P=0.0467), male (P=0.031), advanced pT stage (P<0.0001), and advanced pN stage (P<0.0001) were significantly associated with poor prognosis (Table 3B).

In multivariate analysis, LY6K status did not reach the statistically significant level as independent prognostic factor for surgically treated ESCC patients enrolled in this study (P=0.4479), while pT and pN stages as well as male gender did, suggesting the relevance of LY6K expression to these clinicopathological factors in esophageal cancer (P=0.0138, 0.0002, and <0.0001, respectively; Table 3B).

TABLE 2A
Association between LY6K-positivity in NSCLC tissues
and patients' characteristics (n = 413)

| | | | LY6K | LY6K | | P-value |
|---|---|---|---|---|---|---|
| | | | strong | weak | LY6K | strong |
| | | Total | positive | positive | absent | vs weak/ |
| | | n = 413 | n = 136 | n = 229 | n = 48 | absent |
| Gender | Male | 284 | 102 | 150 | 32 | NS |
| | Female | 129 | 34 | 79 | 16 | |
| Age | <65 | 202 | 72 | 109 | 21 | NS |
| (years) | >=65 | 211 | 64 | 120 | 27 | |
| Histolog- | ADC | 259 | 72 | 152 | 35 | |
| ical type | SCC | 113 | 48 | 56 | 9 | 0.0049⁺ |
| | Others | 41 | 16 | 21 | 4 | |
| pT factor | T1 + T2 | 298 | 97 | 165 | 36 | |
| | T3 + T4 | 115 | 39 | 64 | 12 | NS |
| pN factor | N0 | 257 | 77 | 146 | 34 | NS |
| | N1 + N2 | 156 | 59 | 83 | 14 | |

ADC, adenocarcinoma; SCC, squamous-cell carcinoma
Others, large-cell carcinoma plus adenosquamous-cell carcinoma
*ADC versus other histology
⁺P < 0.05 (Fisher's exact test)
NS, no significance

TABLE 2B
Cox's proportional hazards model analysis of prognostic
factors in patients with NSCLCs

| | | | | |
|---|---|---|---|---|
| | Hazards | | | |
| Variables | ratio | 95% CI | Unfavorable/Favorable | P-value |

Univariate analysis

| | | | | |
|---|---|---|---|---|
| LY6K | 1.545 | 1.161-2.056 | Strong(+)/Weak(+) | 0.0028* |
| | | | or (−) | |
| Age (years) | 1.471 | 1.105-1.956 | 65>=/<65 | 0.0081* |
| Gender | 1.664 | 1.201-2.306 | Male/Female | 0.0022* |
| Histological | 1.458 | 1.099-1.934 | others/ADC¹ | 0.0090* |
| type | | | | |
| pT factor | 1.987 | 1.480-2.667 | T3 + T4/T1 + T2 | <0.0001* |
| pN factor | 2.940 | 2.195-3.937 | N1 + N2/N0 | <0.0001* |

Multivariate analysis

| | | | | |
|---|---|---|---|---|
| LY6K | 1.414 | 1.056-1.893 | Strong(+)/Weak(+) | 0.0201* |
| | | | or (−) | |
| Age (years) | 1.921 | 1.433-2.573 | 65>=/<65 | <0.0001* |
| Gender | 1.552 | 1.083-2.224 | Male/Female | 0.0166* |
| Histological | 1.226 | 0.926-1.731 | others/ADC¹ | 0.1399 |
| type | | | | |
| pT factor | 1.784 | 1.320-2.411 | T3 + T4/T1 + T2 | 0.0002* |
| pN factor | 3.239 | 2.386-4.398 | N1 + N2/N0 | <0.0001* |

¹ADC, adenocarcinoma
*P < 0.05

TABLE 3A
Association between LY6K-positivity in esophageal cancer tissues
and patients' characteristics (n = 271)

| | | | LY6K | LY6K | | P-value |
|---|---|---|---|---|---|---|
| | | | strong | weak | LY6K | strong |
| | | Total | positive | positive | absent | vs weak/ |
| | | n = 271 | n = 176 | n = 81 | n = 14 | absent |
| Gender | Male | 245 | 160 | 74 | 11 | NS |
| | Female | 26 | 16 | 7 | 3 | |
| Age | <65 | 175 | 111 | 58 | 6 | NS |
| (years) | >=65 | 96 | 65 | 23 | 8 | |
| pT factor | T1 − T2 | 126 | 73 | 43 | 10 | 0.0242* |
| | T3 + T4 | 145 | 103 | 38 | 4 | |
| pN factor | N0 | 101 | 60 | 33 | 8 | NS |
| | N1 + N2 | 170 | 116 | 48 | 6 | |

*P < 0.05 (Fisher's exact test)
NS, no significance

TABLE 3B
Cox's proportional hazards model analysis of prognostic
factors in patients with esophageal cancer

| | | | | |
|---|---|---|---|---|
| | Hazards | | | |
| Variables | ratio | 95% CI | Unfavorable/Favorable | P-value |

Univariate analysis

| | | | | |
|---|---|---|---|---|
| LY6K | 1.421 | 1.005-2.010 | Strong(+)/Weak(+) or (−) | 0.0467* |
| Age | 1.023 | 0.734-1.426 | 65>=/<65 | NS |
| (years) | | | | |
| Gender | 3.145 | 1.472-6.720 | Male/Female | 0.031* |
| pT factor | 2.686 | 1.905-3.786 | T3 − T4/T1 − T2 | <0.0001* |
| pN factor | 3.901 | 2.597-5.859 | N1 + N2/N0 | <0.0001* |

Multivariate analysis

| | | | | |
|---|---|---|---|---|
| LY6K | 1.234 | 0.871-1.749 | Strong(+)/Weak(+) or (−) | N.S. |
| Gender | 2.605 | 1.216-5.582 | Male/Female | 0.0138* |
| pT factor | 1.964 | 1.376-2.804 | T3 − T4/T1 − T2 | 0.0002* |
| pN factor | 3.004 | 1.970-4.580 | N1 + N2/N0 | <0.0001* |

*P < 0.05
NS, no significance

### Example 4

Serum Levels of LY6K in Patients with NSCLC or ESCC.

Since the in vitro findings had suggested that LY6K could be secreted into extra cellular space (FIG. 1D, right panel), it was examined whether LY6K was secreted into serum from patients with NSCLC or ESCC in order to validate its potential as a novel serum biomarker. ELISA experiments detected LY6K in serological samples from the great majority of the 193 patients with lung or esophageal cancer.

The mean (+/−1SD) of serum LY6K in 112 lung cancer patients was 331.3+/−739.3 pg/ml and those in 81 ESCC patients were 209.3+/−427.4 pg/ml. In contrast, the mean (+/−1SD) serum levels of LY6K in 74 healthy individuals were 34.2+/−65.3 pg/ml, and those in 65 patients with COPD, who were current and/or former smokers, were 54.4+/−233.8 pg/ml.

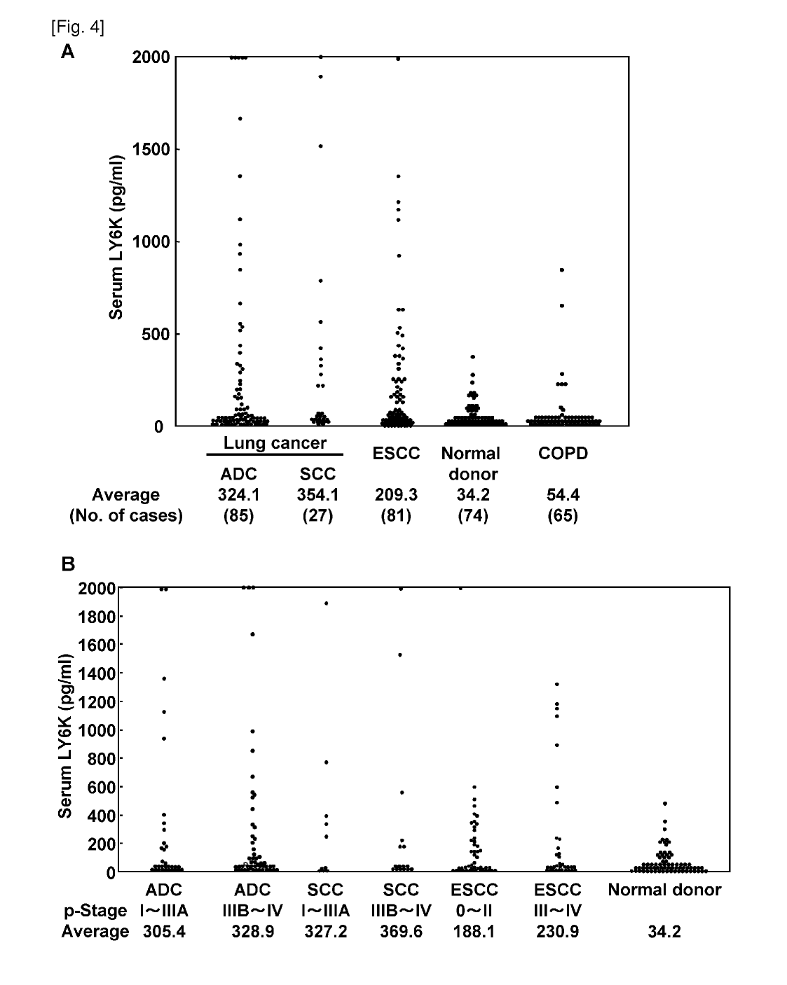

The levels of serum LY6K protein were significantly higher in lung or esophageal cancer patients than in healthy donors (between lung ADC patients and healthy individuals, P<0.0001; between lung SCCs and healthy individuals, P=0.0145; between ESCCs and healthy individuals, P<0.0001; Mann-Whitney U test), while the difference between healthy individuals and COPD patients was not significant (P=0.5325; FIG. 4A).

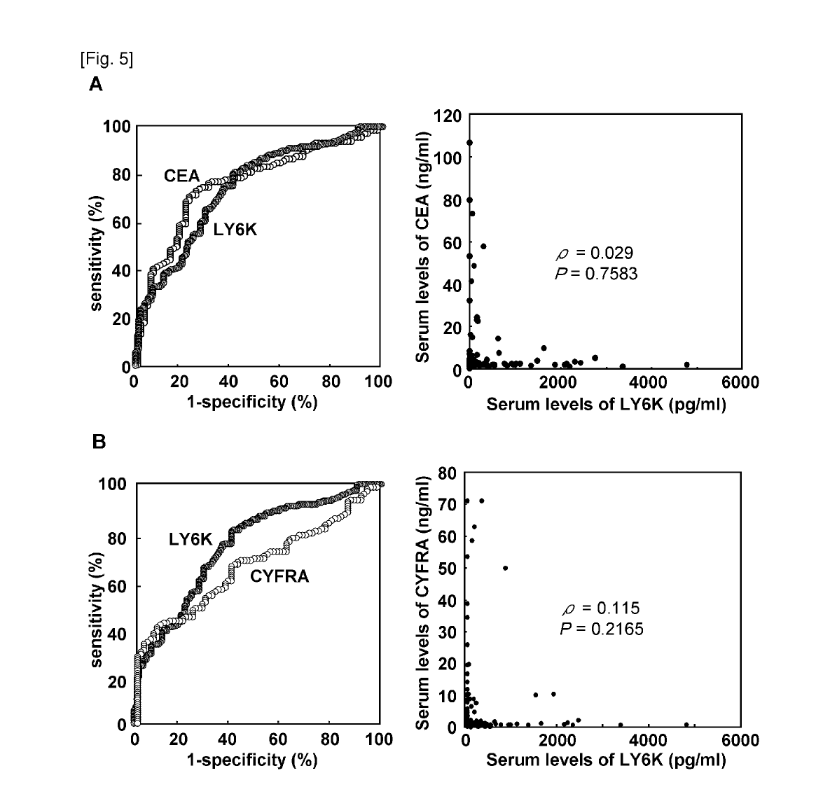

According to histological types of lung cancer, the mean (+/−1SD) serum levels of LY6K were 324.1+/−737.4 pg/ml in 85 ADC patients and those in 27 SCC patients were 354.1+/−758.8 pg/ml; the differences between the two histologic types were not significant. High levels of serum LY6K were detected even in patients with earlier-stage tumors (FIG. 4B). Using receiver-operating characteristic (ROC) curves drawn with the data of these 193 lung or esophageal cancer patients and 74 healthy donors (FIG. 5A and B), the cut-off level in this assay was set to provide optimal diagnostic accuracy and likelihood ratios (minimal false negative and false positive results) for LY6K, i.e., 157.0 pg/ml with a sensitivity of 33.2% (64/193) and a specificity of 4.1% (3/74).

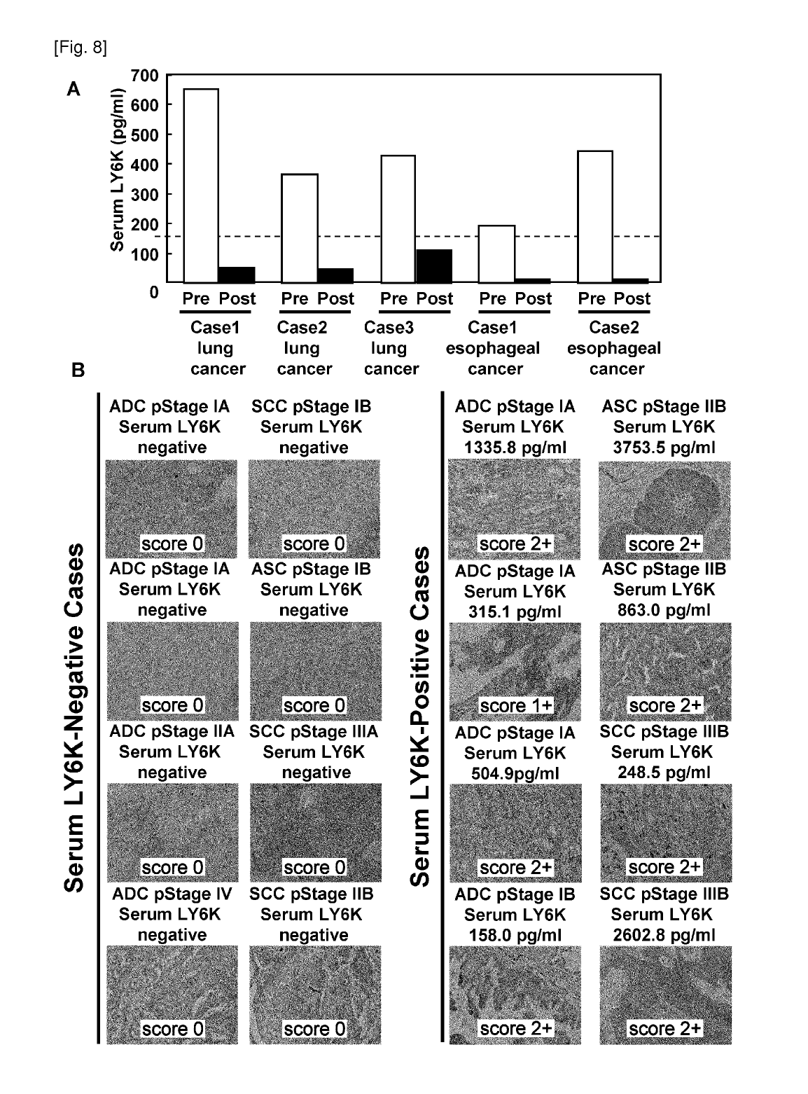

According to tumor histology, the proportions of the serum LY6K-positive cases were 31.8% for ADC (27 of 85), 40.7% for SCC (11 of 27), and 32.1% for ESCC (26 of 81). The proportions of the serum LY6K-positive cases were 9.2% (6/65) for COPD. ELISA experiments were then performed using paired preoperative and post-operative (2 months after the surgery) serum samples from lung cancer and ESCC patients to monitor the levels of serum LY6K in the same patients. The concentration of serum LY6K was significantly reduced after surgical resection of primary tumors (FIG. 8A). The serum LY6K values were further compared with the expression levels of LY6K in primary tumors in the same set of 16 NSCLC cases whose serum had been collected before surgery (eight patients with LY6K-positive tumors and eight with LY6K-negative tumors). The levels of serum LY6K showed good correlation with the expression levels of LY6K in primary tumor (FIG. 8B). The results independently support the high specificity and the great potentiality of serum LY6K as a biomarker for detection of cancer at an early stage and for monitoring of the relapse of the disease.

### Example 5

Comparison of LY6K with CEA and CYFRA 21-1 as Tumor Markers.

To evaluate the feasibility of using serum LY6K level as a tumor-detection biomarker, serum levels of two conventional tumor markers (CEA and CYFRA 21-1 for NSCLC patients) were also measured by ELISA, using both in the same set of serum samples from cancer patients and control individuals. ROC analyses determined the cut off value of CEA for NSCLC detection to be 2.5 ng/ml (with a sensitivity of 39.8% and a specificity of 94.6%; FIG. 5A).

As shown in FIG. 5A, the correlation coefficient between serum LY6K and CEA values was not significant (Spearman rank correlation: rho=0.029, P=0.7583), indicating that measuring both markers in serum can improve overall sensitivity for detection of NSCLC to 61.6%. False-positive results for either of the two tumor-markers among normal volunteers (control group) accounted for 9.5%, while the false-positive rates for CEA and LY6K in the same control group were 4.1% and 5.4%, respectively. According to tumor histology, the sensitivity of the combination of serum LY6K and CEA as a tumor detection marker was 64.7% for ADC and 51.6% for SCC, suggesting the usefulness of this combination for ADC detection.

ROC analyses for the patients with NSCLC determined the cut-off value of CYFRA 21-1 as 2.0 pg/ml, with a sensitivity of 39.8% and a specificity of 97.2% (FIG. 5B). The correlation coefficient between serum LY6K and CYFRA 21-1 values was not significant (Spearman rank correlation: rho=0.115, P=0.2165), also indicating that measurement of serum levels of both markers can improve overall sensitivity for detection of NSCLC to 59.8%; for diagnosing NSCLC, the sensitivity of CYFRA 21-1 alone was 39.8%. False-positive cases for either of the two tumor markers among normal volunteers (control group) were 6.8%, although the false-positive rates for CYFRA 21-1 in the same control group were 2.7%. According to tumor histology, the sensitivity of the combination of serum LY6K and CYFRA 21-1 for the detection of tumors was 56.5% for ADC and 70.4% for SCC, indicating the usefulness of this combination for SCC detection.

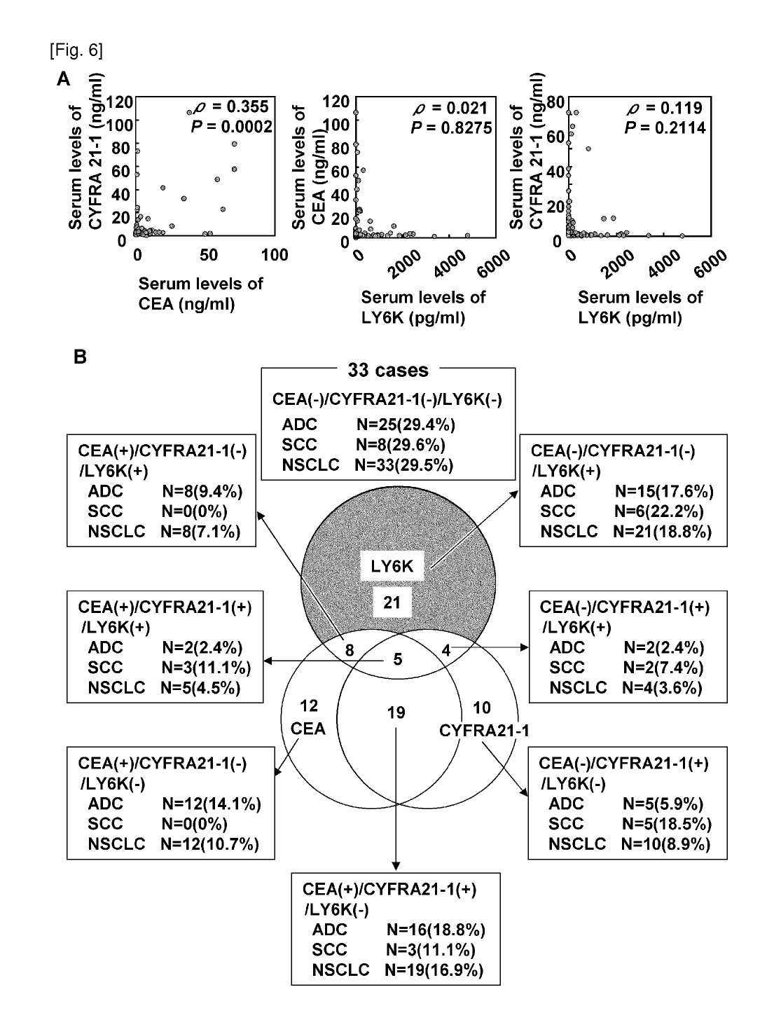

As shown in FIG. 6A (left and middle panels), the correlation coefficient between serum CEA and CYFRA 21-1 values was significant (Spearman rank correlation: rho=0.355, P=0.0002), whereas the correlation between serum LY6K and CEA values was not significant (Spearman rank correlation: rho=0.021, P=0.8275), indicating that measuring both markers in serum can improve overall sensitivity for detection of NSCLC to 61.6%. False-positive results for either of the two tumor-markers among normal volunteers (control group) accounted for 9.5%, while the false-positive rates for CEA and LY6K in the same control group were 4.1% and 5.4%, respectively. According to tumor histology, the sensitivity of the combination of serum LY6K and CEA as a tumor detection marker was 64.7% for ADC and 51.6% for SCC, suggesting the usefulness of this combination for ADC detection.

The correlation coefficient between serum LY6K and CYFRA 21-1 values for NSCLC patients was not significant (Spearman rank correlation: rho=0.119, P=0.2114; Supplementary FIG. 6A, right panel), also indicating that measurement of serum levels of both markers can improve overall sensitivity for detection of NSCLC to 59.8%; for diagnosing NSCLC, the sensitivity of CYFRA 21-1 alone was 33.9%. False-positive cases for either of the two tumor markers among normal volunteers (control group) were 6.8%, although the false-positive rates for CYFRA 21-1 in the same control group were 2.7%. According to tumor histology, the sensitivity of the combination of serum LY6K and CYFRA 21-1 for the detection of tumors was 56.5% for ADC and 70.4% for SCC, indicating the usefulness of this combination for SCC detection. Combination of LY6K with both CEA and CYFRA 21-1 indicated that 21 of 54 (38.9%) NSCLC patients who were negative for both CEA and CYFRA 21-1, were diagnosed as LY6K-positive (FIG. 6B).

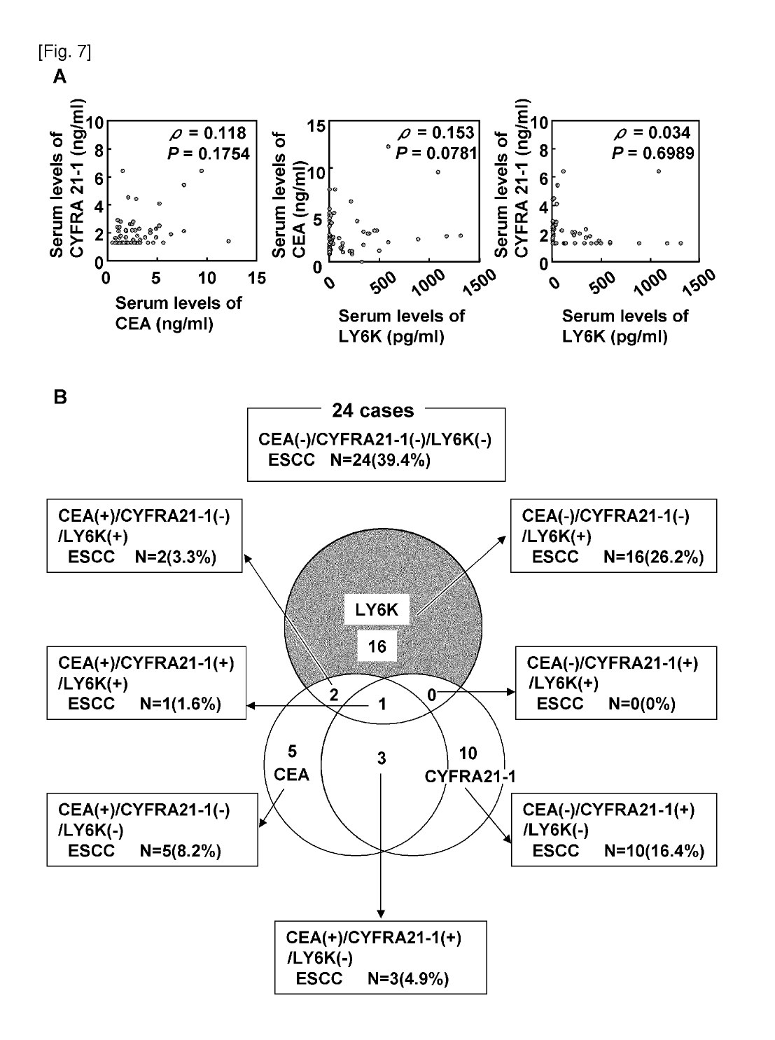

Serum levels of CEA and CYFRA 21-1 were further measured by ELISA in the same set of serum samples from ESCC patients (FIG. 7A). The correlation coefficient between serum LY6K and CEA values for ESCC patients was not significant (Spearman rank correlation: rho=0.153, P=0.0781; FIG. 7A middle panel), indicating that measuring both markers in serum can improve overall sensitivity for detection of ESCC to 44.3%, whereas the sensitivity of CEA alone was 18.0%. The correlation between serum LY6K and CYFRA 21-1 values for ESCC patients was also not significant (Spearman rank correlation: rho=0.034, P=0.6989; FIG. 7A, right panel). A combined assay for both LY6K and CYFRA 21-1 classified 52.5% of ESCC patients as positive, while the sensitivity of CYFRA 21-1 alone was 23.0%. Combination of LY6K with both CEA and CYFRA 21-1 indicated that 16 of 40 (40.0%) ESCC patients who were negative for both CEA and CYFRA 21-1, were diagnosed as LY6K-positive (FIG. 7B). The data clearly suggest that serum LY6K levels were also high in certain proportion of cancer patients that could not be diagnosed by the combination of CEA and CYFRA 21-1.

### Example 6

Effect of LY6K-Small Interfering RNAs on Growth of NSCLC Cells and Esophageal Cancer Cells.

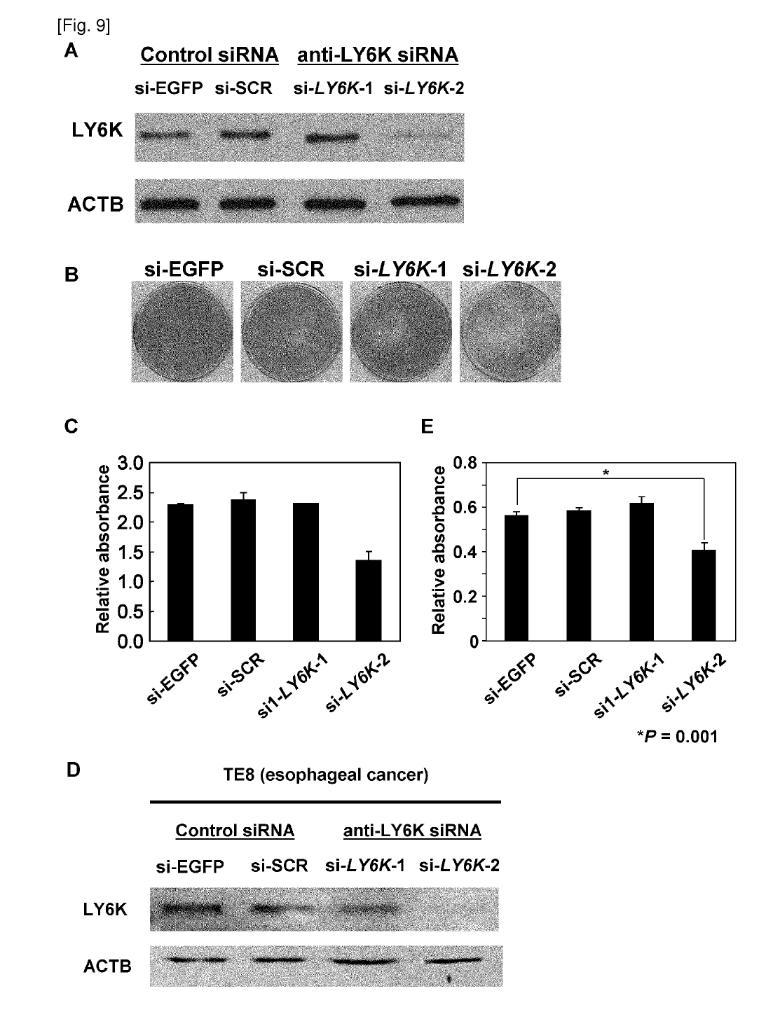

To assess whether LY6K plays a role in growth or survival of lung-cancer cells, plasmids to express siRNA against LY6K (si-LY6K-1 and -2), along with two different control plasmids (siRNAs for EGFP and SCR) were designed, constructed, and transfected into lung cancer (RERF-LC-AI and LC319) and esophageal cancer (TE8) cells to suppress expression of endogenous LY6K (representative data of RERF-LC-AI and TE8 was shown in FIG. 9). The amount of LY6K protein in the cells transfected with si-LY6K-2 was significantly decreased in comparison with cells transfected with any of the two control siRNAs or si-LY6K-1 (FIG. 9A and D). In accordance with its suppressive effect on protein levels of LY6K, transfected si-LY6K-2 caused significant decreases in colony numbers and cell viability measured by colony-formation (FIG. 9B) and MTT assays (FIG. 9C and E).

Discussions

As demonstrated herein, LY6K is expressed only in testis among the normal tissues examined and is highly expressed in 88.2% of surgically resected samples from NSCLC patients and in 95.1% of those from ESCC patients. The LY6K over-expression is associated with the shorter cancer-specific survival period. Suppression of LY6K expression with siRNA effectively suppresses growth of lung and esophageal cancer cells that expressed LY6K. These combined results strongly suggest that LY6K is likely to be associated with highly malignant phenotype of those tumors. Since LY6K is considered to be the cancer-testis antigens, LY6K appears to be a good target for cancer immunotherapy.

It was also found that LY6K protein is secreted into serum from patients with lung cancer or esophageal cancer that strongly expressed LY6K. Due to the fact that concentration of serum LY6K was dramatically reduced after surgical resection of primary tumors and the levels of serum LY6K showed good correlation with the expression levels of LY6K in primary tumor tissue in the same patients, positivity of serum LY6K appears to be considerably correlated with the presence of primary tumors. Interestingly, the correlation coefficient between serum LY6K and CEA or CYFRA 21-1 values was not significant, whereas the correlation coefficient between serum CEA and CYFRA 21-1 values was significant. In fact, 38.9-40.0% of NSCLC and ESCC patients who were negative for both CEA and CYFRA 21-1, were diagnosed to be positive for LY6K (FIG. 6B and 7B). An assay combining both LY6K and CEA/CYFRA 21-1 increased the sensitivity such that 64.7-70.4% of the patients with NSCLC and 52.5% of ESCC were diagnosed as positive, whereas 6.8-9.5% of healthy volunteers were falsely diagnosed as positive. On the other hand, the sensitivity of the combination of conventional serum tumor marker, CEA and CYFRA 21-1 in the same set of serum samples was 51.8% for NSCLC (53.0% for ADC and 48.1% for SCC) and 34.4% for ESCC, while false-positive cases for either of the two tumor markers among normal volunteers (control group) were 6.8% (FIG. 6B and 7B). Although additional validation with a larger set of serum samples covering various clinical stages will be necessary, the data presented here sufficiently demonstrate a potential clinical application of LY6K itself as a serologic/histochemical biomarker for lung and esophageal cancers. It should be also noted that activation of LY6K was observed in more than half of a series of other types of cancers such as cervical carcinomas (data not shown), suggesting its diagnostic and therapeutic application to a wide range of tumors.

### INDUSTRIAL APPLICABILITY

The gene expression analysis of lung cancer and/or esophageal cancer described herein, obtained through a combination of laser-capture dissection and genome-wide cDNA microarray, has identified LY6K genes as targets for cancer prevention and therapy. Based on the expression of LY6K, the present invention provides molecular diagnostic markers for identifying and detecting cancer, particularly lung cancer and/or esophageal cancer.

The methods described herein are also useful in the identification of additional molecular targets for prevention, diagnosis and treatment of cancers such as lung cancer and/or esophageal cancer. The data reported herein add to a comprehensive understanding of lung cancer and/or esophageal cancer, facilitate development of novel diagnostic strategies, and provide clues for identification of molecular targets for therapeutic drugs and preventative agents. Such information contributes to a more profound understanding of lung and/or esophageal tumorigenesis, and provides indicators for developing novel strategies for diagnosis, treatment, and ultimately prevention of lung cancer and/or esophageal cancer.

Furthermore, the methods described herein are also useful in diagnosis of cancer, including lung and esophageal cancers, as well as the prognosis of the patients with these diseases. Moreover, the data reported here is also provide a likely candidate for development of therapeutic approaches for cancer including lung and esophageal cancers.

All publications, patent applications, patents and other references mentioned herein are incorporated by reference in their entirety. However, nothing herein should be construed as an admission that the invention is not entitled to antedate such disclosure by virtue of prior invention. While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Further advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1. A method of diagnosing esophageal cancer or a predisposition for developing esophageal cancer in a subject, including the step of determining a level of expression of LY6K in a biological sample from a patient, wherein an increase in said sample expression level as compared to a normal control expression level of said gene indicates that said subject suffers from or is at risk of developing esophageal cancer.

2. The method of claim 1, wherein said sample expression level is at least 10% greater than said normal control level.

3. The method of claim 1, wherein said esophageal cancer is esophageal squamous-cell carcinoma.

4. The method of claim 1, wherein said biological sample comprises an epithelial cell.

5. The method of claim 1, wherein said biological sample comprises an esophageal cancer cell.

6. The method of claim 1, wherein said biological sample comprises an epithelial cell from an esophageal cancer.

7. The method of claim 1, wherein gene expression level is determined by a method selected from the group consisting of:
(a) detecting mRNA of LY6K,
(b) detecting a protein encoded by LY6K, and
(c) detecting a biological activity of a protein encoded by LY6K.

8. A method of screening for a compound for treating or preventing lung cancer or esophageal cancer, said method comprising the steps of:
(a) contacting a test compound with a polypeptide encoded by LY6K;
(b) detecting the binding activity between the polypeptide and the test compound; and
(c) selecting the test compound that binds to the polypeptide.

9. A method of screening for a compound for treating or preventing lung cancer or esophageal cancer, said method comprising the steps of:
(a) contacting a candidate compound with a cell expressing LY6K; and
(b) selecting the candidate compound that reduces the expression level of LY6K, as compared to an expression level detected in the absence of the candidate compound.

10. The method of claim 9, wherein said cell comprises a lung cancer cell or an esophageal cancer cell.

11. A method of screening for a compound for treating or preventing lung cancer or esophageal cancer, said method comprising the steps of:
(a) contacting a test compound with a polypeptide encoded by LY6K;
(b) detecting the biological activity of the polypeptide of step (a); and
(c) selecting the test compound that suppresses the biological activity of the polypeptide encoded by LY6K, as compared to the biological activity of said polypeptide detected in the absence of the test compound.

12. The method of claim 11, wherein the biological activity of the polypeptide is cell proliferative activity.

13. A method of screening for compound for treating or preventing lung cancer or esophageal cancer, said method comprising the steps of:
(a) contacting a candidate compound with a cell into which a vector, comprising the transcriptional regulatory region of LY6K and a reporter gene that is expressed under the control of the transcriptional regulatory region, has been introduced;
(b) measuring the expression level or activity of said reporter gene; and
(c) selecting the candidate compound that reduces the expression level or activity of said reporter gene, as compared to an expression level or activity detected in the absence of the candidate compound.

14. A method of treating or preventing esophageal cancer in a subject comprising administering to said subject an antisense composition, said antisense composition comprising a nucleotide sequence complementary to a coding sequence of LY6K.

15. A method for treating or preventing esophageal cancer in a subject comprising the step of administering to said subject a pharmaceutically effective amount of an antibody, or an immunologically active fragment thereof, that binds to a protein encoded by LY6K.

16. A composition for treating or preventing esophageal cancer, said composition comprising a pharmaceutically effective amount of an antisense polynucleotide against LY6K.

17. A composition for treating or preventing esophageal cancer, said composition comprising a pharmaceutically effective amount of an antibody or an immunologically active fragment thereof that binds to a protein encoded by LY6K.

18. A method for diagnosing cancer in a subject, comprising the steps of:
(a) collecting a blood sample from a subject to be diagnosed;
(b) determining a level of LY6K in the blood sample;
(c) comparing the LY6K level determined in step (b) with that of a normal control; and
(d) judging that a high LY6K level in the blood sample, as compared to the normal control, indicates that the subject suffers from cancer.

19. The method of claim 18, wherein the cancer is esophageal and/or lung cancer.

20. The method of claim 18, wherein the blood sample is selected from the group consisting of whole blood, serum, and plasma.

21. The method of claim 18, wherein the LY6K level is determined by detecting the LY6K protein in the serum.

22. The method of claim 21, wherein the LY6K protein is detected by immunoassay.

23. The method of claim 22, wherein the immunoassay comprises a step for binding the LY6K protein with an antibody which binds the LY6K protein at the amino acid sequence of SEQ ID NO: 18 or SEQ ID NO: 19 of the protein.

24. The method of claim 22, wherein the immunoassay is an ELISA.

25. The method of claim 24, wherein the ELISA is sandwich method.

26. The method of claim 18, wherein said method further comprises:
(e) determining the level of one or more other cancer-associated proteins in the blood sample;
(f) comparing the protein level(s) determined in step (e) with that of a normal control; and
(g) judging that high level(s) of other cancer-associated proteins in the blood sample when compared to the normal control indicates that the subject suffers from cancer.

27. The method of claim 26, wherein said the cancer-associated protein is either or both of CEA and CYFRA 21-1.

28. A kit for detecting a cancer, wherein the kit comprises:
(a) an immunoassay reagent for determining a level of LY6K in a blood sample; and
(b) a positive control sample for LY6K.

29. The kit of claim 28, wherein the cancer is esophageal and/or lung cancer.

30. The kit of claim 29, wherein the positive control sample is positive for LY6K.

31. The kit of claim 30, wherein the positive control sample is liquid form.

32. The kit of claim 31, wherein the positive control sample is blood sample which comprises a higher than normal level of LY6K.

33. The kit of claim 28, wherein the immunoassay reagent is antibody which recognizes amino acid sequence comprising SEQ ID NO: 18 or 19.

34. The kit of claim 28, which further comprises:
(c) an immunoassay reagent for determining the level of one or more other cancer-associated proteins in a blood sample; and
(d) a positive control for the other cancer-associated proteins.

35. The kit of claim 34, wherein said cancer-associated protein is either or both of CEA and CYFRA 21-1.

36. A method for assessing the prognosis of a patient with cancer, which method comprises the steps of:
(a) detecting the expression level of an LY6K gene in a patient-derived biological sample;
(b) comparing the detected expression level to a control level; and
(c) determining the prognosis of the patient based on the comparison of (b).

37. The method of claim 36, wherein the control level is a good prognosis control level and an increase of the expression level as compared to the control level is determined as poor prognosis.

38. The method of claim 37, wherein the increase is at least 10% greater than said control level.

39. The method of claim 36, wherein said expression level is determined by any one method selected from the group consisting of:
(a) detecting mRNA of the LY6K gene;
(b) detecting the LY6K protein; and
(c) detecting the biological activity of the LY6K protein.

40. The method of claim 36, wherein said expression level is determined by detecting hybridization of a probe to a gene transcript of the LY6K gene.

41. The method of claim 40, wherein the hybridization step is carried out on a DNA array.

42. The method of claim 36, wherein said expression level is determined by correlating the binding of an antibody against the LY6K protein with the expression level of the LY6K gene.

43. The method of claim 42, wherein the antibody recognizes amino acid sequence comprising SEQ ID NO: 18 or 19.

44. The method of claim 36, wherein said biological sample comprises sputum or blood.

45. The method of claim 36, wherein the cancer is esophageal and/or lung cancer.

46. A kit for assessing the prognosis of a patient with cancer, which comprises a reagent selected from the group consisting of:
(a) a reagent for detecting mRNA of an LY6K gene;
(b) a reagent for detecting an LY6K protein; and
(c) a reagent for detecting the biological activity of an LY6K protein.

47. The kit of claim 46, wherein the reagent is an antibody against the LY6K protein.

48. The kit of claim 47, wherein the antibody recognizes amino acid sequence comprising SEQ ID NO: 18 or 19.

49. The kit of claim 46, wherein the cancer is esophageal and/or lung cancer.

50.-66. (canceled)


SEQUENCE LISTING (19 sequences)

SEQ ID NO: 1 - DNA, 1735 bp, Homo sapiens
  CDS (242)..(913)
gttatcagaggtgagcccgtgctcttcagcggagaagatcccctacctggccgccggccactttctgtgggccgtggggtcctcaaggagacggcccttgggctcaggggctgcgtttccacacgcgcctttcccagggctcccgcgcccgttcctgcctggccgccggccgctccaacagcagcacaaggcgggactcagaaccggcgttcagggccgccagcggccgcgaggccctgagatgaggctccaaagaccccgacaggccccggcgggtgggaggcgcgcgccccggggcgggcggggctccccctaccggccagacccggggagaggcgcgcggaggctgcgaaggttccagaagggcggggagggggcgccgcgcgctgaccctccctgggcaccgctggggacgatggcgctgctcgccttgctgctggtcgtggccctaccgcgggtgtggacagacgccaacctgactgcgagacaacgagatccagaggactcccagcgaacggacgagggtgacaatagagtgtggtgtcatgtttgtgagagagaaaacactttcgagtgccagaacccaaggaggtgcaaatggacagagccatactgcgttatagcggccgtgaaaatatttccacgttttttcatggttgcgaagcagtgctccgctggttgtgcagcgatggagagacccaagccagaggagaagcggtttctcctggaagagcccatgcccttcttttacctcaagtgttgtaaaattcgctactgcaatttagaggggccacctatcaactcatcagtgttcaaagaatatgctgggagcatgggtgagagctgtggtgggctgtggctggccatcctcctgctgctggcctccattgcagccggcctcagcctgtcttgagccacgggactgccacagactgagccttccggagcatggactcgctccagaccgttgtcacctgttgcattaaacttgttttctgttgattacctcttggtttgacttcccagggtcttgggatgggagagtggggatcaggtgcagttggctcttaaccctcaagggttctttaactcacattcagaggaagtccagatctcctgagtagtgattttggtgacaagtttttctctttgaaatcaaaccttgtaactcatttattgctgatggccactcttttccttgactcccctctgcctctgagggcttcagtattgatggggagggaggcctaagtaccactcatggagagtatgtgctgagatgcttccgacctttcaggtgacgcaggaacactgggggagtctgaatgattggggtgaagacatccctggagtgaaggactcctcagcatggggggcagtggggcacacgttagggctgcccccattccagtggtggaggcgctgtggatggctgcttttcctcaacctttcctaccagattccaggaggcagaagataactaattgtgttgaagaaacttagacttcacccaccagctggcacaggtgcacagattcataaattcccacacgtgtgtgttcaacatctgaaacttaggccaagtagagagcatcagggtaaatggcgttcatttctctgttaagatgcagccatccatggggagctgagaaatcagactcaaagttccaccaaaaacaaatacaaggggacttcaaaagttcacgaaaaaattgaattaaaagataaaaattaa

SEQ ID NO: 2 - protein, 223 aa, Homo sapiens
MRLQRPRQAPAGGRRAPRGGRGSPYRPDPGRGARRLRRFQKGGEGAPRADPPWAPLGTMALLALLLVVALPRVWTDANLTARQRDPEDSQRTDEGDNRVWCHVCERENTFECQNPRRCKWTEPYCVIAAVKIFPRFFMVAKQCSAGCAAMERPKPEEKRFLLEEPMPFFYLKCCKIRYCNLEGPPINSSVFKEYAGSMGESCGGLWLAILLLLASIAAGLSLS

SEQ ID NO: 3 - DNA, 22 bp, Artificial
  An artificially synthesized primer sequence for RT-PCR
attcgctactgcaatttagagg

SEQ ID NO: 4 - DNA, 23 bp, Artificial
  An artificially synthesized primer sequence for RT-PCR
gtttaatgcaacaggtgacaacg

SEQ ID NO: 5 - DNA, 21 bp, Artificial
  An artificially synthesized primer sequence for RT-PCR
gaggtgatagcattgctttcg

SEQ ID NO: 6 - DNA, 21 bp, Artificial
  An artificially synthesized primer sequence for RT-PCR
caagtcagtgtacaggtaagc

SEQ ID NO: 7 - DNA, 23 bp, Artificial
  An artificially synthesized primer sequence for RT-PCR
agggtgacaatagagtgtggtgt

SEQ ID NO: 8 - DNA, 19 bp, Artificial
  An artificially synthesized target sequence for siRNA
gaagcagcacgacttcttc

SEQ ID NO: 9 - DNA, 19 bp, Artificial
  An artificially synthesized target sequence for siRNA
gcgcgctttgtaggattcg

SEQ ID NO: 10 - DNA, 21 bp, Artificial
  A target sequence for siRNA
aacctgactgcgagacaacga

SEQ ID NO: 11 - DNA, 21 bp, Artificial
  A target sequence for siRNA
aaggaggtgcaaatggacaga

SEQ ID NO: 12 - DNA, 55 bp, Artificial
  An artificially synthesized oligonucleotide for siRNA
tcccaacctgactgcgagacaacgattcaagagatcgttgtctcgcagtcaggtt

SEQ ID NO: 13 - DNA, 55 bp, Artificial
  An artificially synthesized oligonucleotide for siRNA
aaaaaacctgactgcgagacaacgatctcttgaatcgttgtctcgcagtcaggtt

SEQ ID NO: 14 - DNA, 51 bp, Artificial
  siRNA hairpin design
aacctgactgcgagacaacgattcaagagatcgttgtctcgcagtcaggtt

SEQ ID NO: 15 - DNA, 55 bp, Artificial
  An artificially synthesized oligonucleotide for siRNA
tcccaaggaggtgcaaatggacagattcaagagatctgtccatttgcacctcctt

SEQ ID NO: 16 - DNA, 55 bp, Artificial
  An artificially synthesized oligonucleotide for siRNA
aaaaaaggaggtgcaaatggacagatctcttgaatctgtccatttgcacctcctt

SEQ ID NO: 17 - DNA, 51 bp, Artificial
  siRNA hairpin design
aaggaggtgcaaatggacagattcaagagatctgtccatttgcacctcctt

SEQ ID NO: 18 - protein, 87 aa, Artificial
  An artificially synthesized LY6K fragment
SPYRPDPGRGARRLRRFQKGGEGAPRADPPWAPLGTMALLALLLVVALPRVWTDANLTARQRDPEDSQRTDEGDNRVWCHVCERENT

SEQ ID NO: 19 - protein, 134 aa, Artificial
  An artificially synthesized LY6K fragment
PRVWTDANLTARQRDPEDSQRTDEGDNRVWCHVCERENTFECQNPRRCKWTEPYCVIAAVKIFPRFFMVAKQCSAGCAAMERPKPEEKRFLLEEPMPFFYLKCCKIRYCNLEGPPINSSVFKEYAGSMGESCGG